(19) United States
(12) Patent Application Publication
(10) Pub. No.: US 2023/0303700 A1
(43) Pub. Date: Sep. 28, 2023

(54) CELL LOCALIZATION SIGNATURE AND IMMUNOTHERAPY

(71) Applicant: Bristol-Myers Squibb Company, Princeton, NJ (US)

(72) Inventors: George C. LEE, North Wales, PA (US); Robin EDWARDS, Newtown, PA (US); Scott ELY, Princeton, NJ (US); Daniel N. COHEN, Princeton, NJ (US); John B. WOJCIK, Princeton, NJ (US); Vipul A. BAXI, Princeton, NJ (US); Dimple PANDYA, Princeton, NJ (US); Jimena TRILLO-TINOCO, Cambridge, MA (US); Benjamin J. CHEN, Cambridge, MA (US); Andrew FISHER, Cambridge, MA (US); Falon GRAY, Cambridge, MA (US)

(73) Assignee: Bristol-Myers Squibb Company, Princeton, NJ (US)

(21) Appl. No.: 18/043,544

(22) PCT Filed: Aug. 31, 2021
(86) PCT No.: PCT/US2021/048514

§ 371 Date: Feb. 28, 2023

Related U.S. Application Data
(60) Provisional application No. 63/072,651, filed on Aug. 31, 2020.

(51) Int. Cl. C07K 16/28; C07K 14/705; G01N 33/574; G01N 33/50

(52) U.S. Cl. CPC C07K 16/2827; C07K 14/70517; C07K 16/2818; G01N 33/505; G01N 33/57492

(57) ABSTRACT

The present disclosure provides methods of identifying a subject suitable for an anti-PD-⅟PD-L1 antagonist therapy comprising measuring assay CD8 localization and PD-L1 expression in a tumor sample obtained from the subject. In some aspects, method further comprises administering (i) an anti-PD-⅟PD-L1 antagonist therapy or (ii) an anti-PD-⅟PD-L1 antagonist and anti-CT-LA-4 antagonist combination therapy to a subject identified as having a tumor exhibiting an excluded CD8 localization phenotype, wherein the tumor is PD-L1 negative.

### CROSS-REFERENCE TO EARLIER FILED APPLICATIONS

This PCT application claims the priority benefit of U.S. Provisional Application No. 63/072,651 filed on Aug. 31, 2020, which is incorporated by reference herein in its entirety.

### FIELD OF THE DISCLOSURE

The present disclosure provides a method for treating a subject afflicted with a tumor using an immunotherapy.

### BACKGROUND OF THE DISCLOSURE

Human cancers harbor numerous genetic and epigenetic alterations, generating neoantigens potentially recognizable by the immune system (Sjoblom et al., Science (2006) 314(5797):268-274). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

In the past decade, intensive efforts to develop specific immune checkpoint pathway inhibitors have begun to provide new immunotherapeutic approaches for treating cancer, including the development of antibodies that block the inhibitory Programmed Death-1 (PD-1)/Programmed Death ligand 1 (PD-L1) pathway such as nivolumab and pembrolizumab (formerly lambrolizumab; USAN Council Statement, 2013) that bind specifically to the PD-1 receptor and atezolizumab, durvalumab, and avelumab that bind specifically to PD-L1.

The immune system and response to immuno-therapy have shown to be complex. Additionally, anti-cancer agents can vary in their effectiveness based on the unique patient characteristics. Accordingly, there is a need for targeted therapeutic strategies that identify patients who are more likely to respond to a particular anti-cancer agent and, thus, improve the clinical outcome for patients diagnosed with cancer.

### SUMMARY OF THE DISCLOSURE

Certain aspects of the present disclosure are directed to a pharmaceutical composition comprising an anti-PD-⅟PD-L1 antagonist for use in a method of treating a human subject afflicted with a tumor, wherein a tumor sample obtained from the subject exhibits: (i) an excluded CD8 localization phenotype, and (ii) a negative PD-L1 expression status. In some aspects, the subject is to be administered an anti-PD-⅟PD-L1 antagonist in combination with an anti-cancer agent. In some aspects, the subject is to be administered an anti-PD-⅟PD-L1 antagonist in combination with an anti-CTLA-4 antagonist.

In some aspects, the tumor sample is a tumor tissue biopsy. In some aspects, the tumor sample is a formalin-fixed, paraffin-embedded tumor tissue or a fresh-frozen tumor tissue.

In some aspects, the CD8 localization is measured by staining the tumor sample with an antibody or an antigen-binding portion thereof that binds CD8. In some aspects, the tumor sample is imaged following the staining with the antibody.

In some aspects, the PD-L1 expression is measured by staining the tumor sample with an antibody or an antigen-binding portion thereof that specifically binds PD-L1. In some aspects, the negative PD-L1 expression status is characterized by a tumor sample wherein less than about 1% of tumor cells express PD-L1. In some aspects, the PD-L1 expression is measured using an IHC assay. In some aspects, the IHC assay comprises an automated IHC assay. In some aspects, the CD8 localization is measured by IHC followed by classification of the CD8 localization in the tumor sample.

In some aspects, the classification is performed by a method comprising: receiving, by at least one processor of a computing device, a plurality of histology images of tumor samples in a plurality of patients; performing, by the at least one processor, an image analysis of the plurality of histology images to obtain a CD8+ T-cell abundance in the tumor parenchyma and stroma in each of the plurality of histology images; training, by the at least one processor, a machine learning algorithm using results of the image analysis and the CD8+ T-cell abundance in the tumor parenchyma and stroma; generating, by the at least one processor, a machine learning feature space comprising a plurality of classifications based on the training; and identifying, by the at least one processor, boundaries between the plurality of classifications in the machine learning feature space.

Certain aspects of the present disclosure are directed to a pharmaceutical composition comprising an anti-PD-⅟PD-L 1 antagonist for use in a method of identifying a human subject suitable for an anti-PD-⅟PD-L1 antagonist therapy, wherein the method comprises (i) measuring an expression of PD-L1 in a tumor sample obtained from the subject, and (ii) measuring CD8 localization in the tumor sample; wherein the CD8 localization is measured by staining the tumor sample with an antibody or an antigen-binding portion thereof that binds CD8, and classification of the CD8 localization in the tumor sample; wherein the classification is performed by a method comprising: receiving, by at least one processor of a computing device, a plurality of histology images of tumor samples in a plurality of patients; performing, by the at least one processor, an image analysis of the plurality of histology images to obtain a CD8+ T-cell abundance in the tumor parenchyma and stroma in each of the plurality of histology images; training, by the at least one processor, a machine learning algorithm using results of the image analysis and the CD8+ T-cell abundance in the tumor parenchyma and stroma; generating, by the at least one processor, a machine learning feature space comprising a plurality of classifications based on the training; and identifying, by the at least one processor, boundaries between the plurality of classifications in the machine learning feature space.

In some aspects, performing the image analysis of the plurality of histology images comprises applying an artificial neural network to the plurality of histology images. In some aspects, the machine-learning algorithm comprises a random forest classifier algorithm. In some aspects, the CD8+ T-cell abundance comprises a graphical representation of a relationship between percentages of the stromal CD8+ T-cells and percentages of the parenchymal CD8+ T-cells with respect to the total number of T-cells present in each of the plurality of histology images. In some aspects, the pharmaceutical composition for use further comprises applying, by the at least one processor of the computing device, a polar coordinate transformation of the graphical representation, resulting in a polar plot; and using the polar plot to train the machine learning algorithm. In some aspects, the plurality of classifications comprises inflamed, desert, excluded, or balanced.

In some aspects, the pharmaceutical composition for use further comprises determining a classification for each of the plurality of histology images based on the machine learning feature space. In some aspects, the pharmaceutical composition for use further comprises validating results from the machine learning feature space by comparing a label for each of the plurality of histology images obtained by at least one pathologist to the classification for each of the plurality of histology images. In some aspects, the pharmaceutical composition for use further comprises: receiving, by the at least one processor of the computing device, an additional histology image; performing an additional image analysis of the additional histology image and obtaining an additional CD8+ T-cell abundance in the tumor parenchyma and stroma in the additional histology image; applying the machine learning algorithm to results from the additional image analysis and the additional CD8+ T-cell abundance; and determining a classification for the additional histology image based on the machine learning feature space.

In some aspects, the CD8 localization is measured by measuring expression of a panel of genes in a tumor sample obtained from the subject.

In some aspects, a subject identified as having an excluded CD8 localization phenotype and a PD-L1 negative tumor is to be administered therapy comprising the anti-PD-⅟PD-L1 antagonist. In some aspects, a subject identified as having an excluded CD8 localization phenotype and a PD-L1 negative tumor is to be administered therapy comprising the anti-PD-⅟PD-L1 antagonist and an anti-CTLA-4 antagonist.

In some aspects, the anti-PD-⅟PD-L1 antagonist comprises an antibody or antigen-binding fragment thereof that specifically binds a target protein selected from programmed death 1 (PD-1; an “anti-PD-1 antibody”) or programmed death ligand 1 (PD-L1; an “anti-PD-L1 antibody”). In some aspects, the anti-PD-⅟PD-L1 antagonist comprises an anti-PD-1 antibody. In some aspects, the anti-PD-1 antibody comprises nivolumab or pembrolizumab.

In some aspects, the anti-PD-⅟PD-L1 antagonist comprises an anti-PD-L1 antibody. In some aspects, the anti-PD-L1 antibody comprises avelumab, atezolizumab, or durvalumab.

In some aspects, the anti-CTLA-4 antagonist comprises an antibody or antigen-binding fragment thereof that specifically binds cytotoxic T-lymphocyte-associated protein 4 (CTLA-4; an “anti-CTLA-4 antibody”). In some aspects, the anti-CTLA-4 antibody comprises ipilimumab.

Certain aspects of the present disclosure are directed to a method of treating a cancer in a human subject, comprising administering an anti-PD-⅟anti-PD-L1 antagonist to a subject, wherein the subject is identified as having a tumor exhibiting: (i) an excluded CD8 localization phenotype; and (ii) a negative PD-L1 expression status. In some aspects, the method further comprises administering an anti-CTLA-4 antagonist.

In some aspects, the excluded CD8 localization phenotype is measured by detecting CD8 expression in a tumor sample obtained from the subject. In some aspects, the excluded CD8 localization phenotype is measured by staining the tumor sample with an antibody or an antigen-binding portion thereof that binds CD8. In some aspects, the CD8 localization is measured by staining the tumor sample with an antibody or an antigen-binding portion thereof that binds CD8 followed by classification of the CD8 localization in the tumor sample; wherein the classification is performed by a method comprising; receiving, by at least one processor of a computing device, a plurality of histology images of tumor samples in a plurality of patients; performing, by the at least one processor, an image analysis of the plurality of histology images to obtain a c CD8+ T-cell abundance in the tumor parenchyma and stroma in each of the plurality of histology images; training, by the at least one processor, a machine learning algorithm using results of the image analysis and the CD8+ T-cell abundance in the tumor parenchyma and stroma; generating, by the at least one processor, a machine learning feature space comprising a plurality of classifications based on the training; and identifying, by the at least one processor, boundaries between the plurality of classifications in the machine learning feature space.

Certain aspects of the present disclosure are directed to a method of identifying a human subject suitable for an anti-PD-⅟PD-L1 antagonist therapy, comprising (i) measuring an expression of PD-L1 in a tumor sample obtained from the subject, and (ii) measuring CD8 localization in the tumor sample; wherein the CD8 localization is measured by staining the tumor sample with an antibody or an antigen-binding portion thereof that binds CD8 followed by classification of the CD8 localization in the tumor sample; wherein the classification is performed by a method comprising: receiving, by at least one processor of a computing device, a plurality of histology images of tumor samples in a plurality of patients; performing, by the at least one processor, an image analysis of the plurality of histology images to obtain a CD8+ T-cell abundance in the tumor parenchyma and stroma in each of the plurality of histology images; training, by the at least one processor, a machine learning algorithm using results of the image analysis and the CD8+ T-cell abundance in the tumor parenchyma and stroma; generating, by the at least one processor, a machine learning feature space comprising a plurality of classifications based on the training; and identifying, by the at least one processor, boundaries between the plurality of classifications in the machine learning feature space.

In some aspects, performing the image analysis of the plurality of histology images comprises applying an artificial neural network to the plurality of histology images. In some aspects, the machine-learning algorithm comprises a random forest classifier algorithm. In some aspects, the CD8+ T-cell abundance comprises a graphical representation of a relationship between percentages of the stromal CD8+ T-cells and percentages of the parenchymal CD8+ T-cells with respect to the total number of T-cells present in each of the plurality of histology images. In some aspects, the method further comprises applying, by the at least one processor of the computing device, a polar coordinate transformation of the graphical representation, resulting in a polar plot; and using the polar plot to train the machine learning algorithm. In some aspects, the plurality of classifications comprises inflamed, desert, excluded, or balanced.

In some aspects, the method further comprises determining a classification for each of the plurality of histology images based on the machine learning feature space. In some aspects, the method further comprises validating results from the machine learning feature space by comparing a label for each of the plurality of histology images obtained by at least one pathologist to the classification for each of the plurality of histology images. In some aspects, the method further comprises receiving, by the at least one processor of the computing device, an additional histology image; performing an additional image analysis of the additional histology image and obtaining an additional CD8+ T-cell abundance in the tumor parenchyma and stroma in the additional histology image; applying the machine learning algorithm to results from the additional image analysis and the additional CD8+ T-cell abundance; and determining a classification for the additional histology image based on the machine learning feature space.

In some aspects, the method further comprises administering the anti-PD-⅟PD-L1 antagonist to a subject identified as having an excluded CD8 localization phenotype and a PD-L1 negative tumor. In some aspects, the method further comprises administering an anti-CTLA-4 antagonist.

In some aspects, the anti-PD-⅟PD-L1 antagonist comprises an antibody or antigen-binding fragment thereof that specifically binds a target protein selected from programmed death 1 (PD-1; an “anti-PD-1 antibody”) or programmed death ligand 1 (PD-L1; an “anti-PD-L1 antibody”). In some aspects, the anti-PD-⅟PD-L1 antagonist is an anti-PD-1 antibody. In some aspects, the anti-PD-1 antibody comprises nivolumab or pembrolizumab. In some aspects, the anti-PD-⅟PD-L1 antagonist comprises an anti-PD-L1 antibody. In some aspects, the anti-PD-L1 antibody comprises avelumab, atezolizumab, or durvalumab. In some aspects, the anti-CTLA-4 antagonist comprises an antibody or antigen-binding fragment thereof that specifically binds cytotoxic T-lymphocyte-associated protein 4 (CTLA-4; an “anti-CTLA-4 antibody”). In some aspects, the anti-CTLA-4 antibody comprises ipilimumab.

In some aspects, the tumor is derived from a cancer selected from the group consisting of hepatocellular cancer, gastroesophageal cancer, melanoma, bladder cancer, lung cancer, kidney cancer, head and neck cancer, colon cancer, pancreatic cancer, prostate cancer, ovarian cancer, urothelial cancer, colorectal cancer, and any combination thereof. In some aspects, the tumor is relapsed. In some aspects, the tumor is refractory. In some aspects, the tumor is locally advanced. In some aspects, the tumor is metastatic.

In some aspects, the administering treats the tumor. In some aspects, the administering reduces the size of the tumor. In some aspects, the size of the tumor is reduced by at least about 10%, about 20%, about 30%, about 40%, or about 50% compared to the tumor size prior to the administration. In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some aspects, the subject exhibits stable disease after the administration. In some aspects, the subject exhibits a partial response after the administration. In some aspects, the subject exhibits a complete response after the administration.

Certain aspects of the present disclosure are directed to a kit for treating a subject afflicted with a tumor, the kit comprising: (a) an anti-PD-⅟PD-L1 antagonist; and (b) instructions for using the anti-PD-⅟PD-L1 antagonist according to a method disclosed herein. In some aspects, the anti-PD-⅟PD-L1 antagonist comprises an anti-PD-1 antibody. In some aspects, the anti-PD-⅟PD-L1 antagonist comprises an anti-PD-L1 antibody. In some aspects, the kit further comprises an anti-CTLA-4 antagonist. In some aspects, the anti-CTLA-4 agonist comprises and anti-CTLA-4 antibody.

In some aspects, the subject exhibits less severe adverse events, as compared to a subject that does not exhibit an excluded CD8 localization phenotype. In some aspects, the subject does not exhibit an adverse event more severe than a grade 1 adverse event, more severe than a grade 2 adverse event, or more severe than a grade 3 adverse event. In some aspects, the subject exhibits fewer adverse events of grade 3 or more severe, as compared to a subject that does not exhibit an excluded CD8 localization phenotype.

### BRIEF DESCRIPTION OF THE DRAWINGS

FIG. 1 illustrates example images of tumor tissue samples with various classifications using CD8+ immunostaining followed by imaging, according to example embodiments.

FIG. 2 is an example diagram illustrating a methodology for image analysis and machine learning-based approaches for training a model for tumor topology classification, according to example embodiments.

FIG. 3 is another example diagram illustrating the methodology for classification of tumor topology using image analysis and machine learning-based approaches, according to example embodiments.

FIG. 4 is a flowchart illustrating the process for training a machine learning algorithm for classification of CD8 tumor topology, according to example embodiments.

FIG. 5 is a flowchart illustrating the process for classifying CD8 tumor topology of a histology image using the trained machine learning algorithm, according to example embodiments.

FIG. 6 is a block diagram of example components of a device according to example embodiments.

FIGS. 7A-7C are graphical representations of overall survival (OS) in patients having PD-L1 negative (PD-L1 expression less than 1%) melanoma (FIGS. 7A-7B) or urothelial carcinoma (FIG. 7C) tumors, following treatment with either an anti-PD-1 antibody (FIGS. 7A and 7C) or a combination of an anti-PD-1 antibody and an anti-CTLA-4 antibody (FIG. 7B). Patients were stratified by CD8 topology as either having an excluded CD8 phenotype (FIGS. 7A-7C), an inflamed CD8 phenotype (FIGS. 7A-7C), or a desert CD8 phenotype (FIG. 7C), as measured using immunohistochemistry followed by machine learning analysis, as described herein. Patients at risk in each group are shown in FIGS. 7A-7B.

### DETAILED DESCRIPTION OF THE DISCLOSURE

Certain aspects of the present disclosure are directed to methods of treating a human subject afflicted with a tumor, comprising administering to the subject an anti-PD-⅟PD-L1 antagonist, wherein a tumor sample obtained from the subject exhibits (i) an excluded CD8 localization phenotype and (ii) a negative PD-L1 expression status (“PD-L1-negative”).

Other aspects of the present disclosure are directed to methods of identifying a subject suitable for an immune-oncology (I-O) therapy, e.g., an anti-PD-⅟PD-L1 antagonist therapy alone or in combination with an anti-CTLA-4 antagonist therapy. In some aspects, the method comprises (i) measuring the expression of PD-L1 in a tumor sample obtained from the subject, and (ii) measuring CD8 expression in the tumor sample; wherein the CD8 expression is measured by immunostaining and imaging followed by classification of the localization CD8 expression in the tumor sample using a machine-learning algorithm. In some aspects, the method further comprises administering an anti-PD-⅟PD-L1 antagonist to a subject identified as having a tumor sample that exhibits (i) an excluded CD8 localization phenotype and (ii) a negative PD-L1 expression status (“PD-L1-negative”).

In some aspects, the method further comprises administering an additional anti-cancer agent. In some aspects, the method further comprises administering an anti-CTLA-4 antagonist.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is understood that wherever aspects are described herein with the language “comprising,” otherwise analogous aspects described in terms of “consisting of” and/or “consisting essentially of” are also provided.

Certain aspects disclosed herein may be implemented in hardware (e.g., circuits), firmware, software, or any combination thereof. Some aspects may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a general purpose computer, as described herein.

For purposes of this discussion, any reference to the term “module” shall be understood to include at least one of software, firmware, or hardware (such as one or more of a circuit, microchip, and device, or any combination thereof), and any combination thereof. In addition, it will be understood that each module may include one, or more than one, component within an actual device, and each component that forms a part of the described module may function either cooperatively or independently of any other component forming a part of the module. Conversely, multiple modules described herein may represent a single component within an actual device. Further, components within a module may be in a single device or distributed among multiple devices in a wired or wireless manner.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values, which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

As used herein, the terms “CD8 localization” and “CD8 topology” are used interchangeably, and refer to the general compartmental distribution of CD8⁺ cells in a sample, e.g., a tumor sample obtained by a subject, using the methods disclosed herein. An “excluded” or “stromal” CD8 localization phenotype refers to a sample wherein the majority or all of the CD8⁺ cells are located outside of the tumor parenchyma. An “inflamed” or “parenchymal” CD8 localization phenotype refers to a sample wherein a multitude of CD8⁺ cells is located within the tumor parenchyma. A “cold” or “desert CD8 localization phenotype refers to a sample wherein there are no CD8⁺ cells detected. CD8 is a marker for CD8⁺ T cells, and thus, in some aspects, CD8 localization is indicative of an immune response to the tumor.

“Administering” refers to the physical introduction of a composition comprising a therapeutic agent to a subject, using any of the various methods and delivery systems known to those skilled in the art. Preferred routes of administration for an immunotherapy, e.g., with anti-PD-1 antibody or the anti-PD-L1 antibody, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime. In some aspects, the methods disclosed herein identify a subject having an excluded CD8 localized phenotype, wherein the subject exhibits less severe adverse events following administration of a composition comprising an anti-PD-⅟PD-L1 antagonist, as compared to a subject that does not exhibit an excluded CD8 localization phenotype. In some aspects, the subject does not exhibit an adverse event more severe than a grade 1 adverse event, more severe than a grade 2 adverse event, or more severe than a grade 3 adverse event. In some aspects, the subject exhibits fewer adverse events of grade 3 or more severe, as compared to a subject that does not exhibit an excluded CD8 localization phenotype. In some aspects, the subject exhibits fewer adverse events of grade 2 or more severe, as compared to a subject that does not exhibit an excluded CD8 localization phenotype. The specific nature of each AE grade level depends on the indication and/or condition. Application of the AE grading system can be found in the Common Terminology Criteria for Adverse Events (CTCAE) v5.0 published by the National Cancer Institute, which is available at ctep.cancer.gov/protocolDevelopment/electronic_applications/ctc.htm#ctc_60, and which is incorporated by reference herein in its entirety.

An “antibody” (Ab) shall include, without limitation, a glycoprotein immunoglobulin, which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V₁) and a light chain constant region. The light chain constant region is comprises one constant domain, C₁. The V_{H} and V_{L} regions can be further subdivided into regions of hypervariability, termed complementarity determining regions (CDRs), interspersed with regions that are more conserved, termed framework regions (FRs). Each V_{H} and V_{L}, comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, the term “anti-PD-1 antibody” includes a full antibody having two heavy chains and two light chains that specifically binds to PD-1 and antigen-binding portions of the full antibody. Non-limiting examples of the antigen-binding portions are shown elsewhere herein.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1, an anti-PD-L1 antibody binds specifically to PD-L1, and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-PD-1 antibody or an anti-PD-L1 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

Antibodies useful in the methods and compositions described herein include, but are not limited to, antibodies and antigen-binding portions thereof that specifically bind a protein selected from the group consisting of Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), Cytotoxic T-Lymphocyte Antigen-4 (CTLA-4), B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, IL-2, B7-H4, Fas ligand, CXCR4, CSF1R, mesothelin, CEACAM-1, CD52, HER2, MICA, MICB, CSF1R, and any combination thereof.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

“PD-L1 negative” as used herein can be interchangeably used with “PD-L1 expression of less than about 1%.” The PD-L1 expression can be measured by any methods known in the art. In some aspects, the PD-L1 expression is measured by an automated immunohistochemistry (IHC). In some aspects, PD-L1 negative tumors can thus have less than about 1% of the tumor cells expressing PD-L1 as measured by an automated IHC. In some aspects, a PD-L1 negative tumor has no tumor cells expressing PD-L1.

As used herein, a PD-1 or PD-L1 “inhibitor,” refers to any molecule capable of blocking, reducing, or otherwise limiting the interaction between PD-1 and PD-L1 and/or the activity of PD-1 and/or PD-L1. In some aspects, the inhibitor is an antibody or an antigen-binding fragment of an antibody. In other aspects, the inhibitor comprises a small molecule.

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, an “immuno-oncology” therapy or an “I-O” therapy refers to a therapy that comprises utilizing an immune response to target and treat a tumor in a subject. As such, as used herein, an I-O therapy is a type of anti-cancer therapy. In some aspects, and I-O therapy comprises administering an antibody or an antigen-binding fragment thereof to a subject. In some aspects, an I-O therapy comprises administering to a subject an immune cell, e.g., a T cell, e.g., a modified T cell, e.g., a T cell modified to express a chimeric antigen receptor or a particular T cell receptor. In some aspects, the I-O therapy comprises administering a therapeutic vaccine to a subject. In some aspects, the I-O therapy comprises administering a cytokine or a chemokine to a subject. In some aspects, the I-O therapy comprises administering an interleukin to a subject. In some aspects, the I-O therapy comprises administering an interferon to a subject. In some aspects, the I-O therapy comprises administering a colony-stimulating factor to a subject.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred aspects of the disclosure, tumor regression can be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for immune-related response patterns.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate’s body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that promotes cancer regression in a subject. In some aspects, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, an increase in overall survival, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some aspects, a therapeutically effective amount or dosage of the drug completely inhibits cell growth or tumor growth, i.e., inhibits cell growth or tumor growth by 100%. The ability of a compound to inhibit tumor growth can be evaluated using an assay described herein. Alternatively, this property of a composition can be evaluated by examining the ability of the compound to inhibit cell growth, such inhibition can be measured in vitro by assays known to the skilled practitioner. In some aspects described herein, tumor regression can be observed and continue for a period of at least about 20 days, at least about 40 days, or at least about 60 days.

The term “biological sample” as used herein refers to biological material isolated from a subject. The biological sample can contain any biological material suitable for determining target gene expression, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one aspect, the sample is a tumor sample. In some aspects, the tumor sample can be obtained from a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

A “tumor sample,” as used herein, refers to a biological sample that comprises tumor tissue. In some aspects, a tumor sample is a tumor biopsy. In some aspects, a tumor sample comprises tumor cells and one or more non-tumor cell present in the tumor microenvironment (TME). For the purposes of the present disclosure, the TME is made up of at least two regions. The tumor “parenchyma” is a region of the TME that includes predominantly tumor cells, e.g., the part (or parts) of the TME that includes the bulk of the tumor cells. The tumor parenchyma does not necessarily consist of only tumor cells, rather other cells such as stromal cells and/or lymphocytes can also be present in the parenchyma. The “stromal” region of the TME includes the adjacent non-tumor cells. In some aspects, the tumor sample comprises all or part of the tumor parenchyma and one or more cells of the stroma. In some aspects, the tumor sample is obtained from the parenchyma. In some aspects the tumor sample is obtained from the stroma. In other aspects, the tumor sample is obtained from the parenchyma and the stroma.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Disclosure

PD-L1 expression has been identified as a biomarker for responsiveness to an anti-PD-1 antibody therapy. The present disclosure surprisingly found that a subpopulation of PD-L1 negative tumors are nonetheless responsive to therapies targeting PD-1 signaling. This was observed for both an anti-PD-1 antibody monotherapy and a combination therapy comprising an anti-PD-1 antibody and an anti-CTLA-4 antibody.

Certain aspects of the present disclosure are directed to methods of treating a human subject afflicted with a tumor, comprising administering to the subject an anti-PD-⅟PD-L1 antagonist, wherein a tumor sample obtained from the subject exhibits (i) an excluded CD8 localization phenotype and (ii) a negative PD-L1 expression status (“PD-L1-negative”).

Other aspects of the present disclosure are directed to methods of identifying a subject suitable for an immune-oncology (I-O) therapy, e.g., an anti-PD-⅟PD-L1 antagonist therapy alone or in combination with an anti-CTLA-4 antagonist therapy. In some aspects, the method comprises (i) measuring the expression of PD-L1 in a tumor sample obtained from the subject, and (ii) measuring CD8 expression in the tumor sample; wherein the CD8 expression is measured by immunostaining and imaging followed by classification of the localization CD8 expression in the tumor sample using a machine-learning algorithm. In some aspects, the method further comprises administering an anti-PD-⅟PD-L1 antagonist to a subject identified as having a tumor sample that exhibits (i) an excluded CD8 localization phenotype and (ii) a negative PD-L1 expression status (“PD-L1-negative”).

In some aspects, the method further comprises administering an additional anti-cancer agent. In some aspects, the method further comprises administering an anti-CTLA-4 antagonist.

In some aspects, the tumor sample obtained from the subject comprises a tumor biopsy. In some aspect, the tumor sample is a formalin-fixed, paraffin-embedded tumor tissue. In some aspects, the tumor sample is a fresh-frozen tumor tissue.

### II.A. Measuring CD8 and PD-L1 Expression

CD8 localization and/or PD-L1 expression in the tumor sample can be measured using any methods known in the art. In some aspects, CD8 expression is measured using a first method, and PD-L1 expression is measured using a second method, wherein the first method and the second method are different. In some aspects, CD8 expression and PD-L1 expression are measured in the same tumor sample. In some aspects, CD8 expression and PD-L1 expression are measured in two different tumor samples obtained from the same subject. In some aspects, CD8 expression and PD-L1 expression are measured in two different tumor samples obtained from the same subject, wherein the two different tumor samples are two sections of the same tumor. In some aspects, CD8 expression and PD-L1 expression are measured in two different tumor samples obtained from the same subject, wherein the two different tumor samples are two adjacent sections of the same tumor.

### II.A.1. CD8 Localization

CD8 localization can be determined using any methods known in the art. In some aspects, the methods comprise directly measuring the localization of CD8 expression, e.g., the location of CD8-expressing cells, in a tumor sample obtained from a subject. In certain aspects, CD8 localization comprises measuring CD8 protein in the tumor sample. In some aspects, CD8 protein is measured by contacting the tumor sample with an antibody or an antigen-binding portion thereof that binds CD8. In some aspects, CD8 localization is measured using an immunostaining assay. In some aspects, the assay comprises an automated immunostaining assay. In other aspects, CD8 localization comprises measuring CD8 mRNA in the tumor sample. In some aspects, CD8 localization is measured using an RNA in situ hybridization assay. In other some aspects, CD8 localization is measured by isolating RNA from the tumor sample, or a subsection thereof, and measuring CD8 expression by a reverse transcriptase PCR reaction (RT-PCR) assay.

In certain aspects, CD8 localization is measured by staining the tumor sample with an antibody or an antigen-binding portion thereof that binds CD8. In some aspects, CD8 localization is measured by staining the tumor sample with an antibody or an antigen-binding portion thereof that binds CD8 and imaging the tumor sample, e.g., preparing one or more histology images of the tumor sample. Imaging of the tumor sample can be done by a human or it can be automated, e.g., competed by a machine. In some aspects, the histology images are analyzed by a human, e.g., a pathologist, and the CD8 expression is characterized by the human. In other aspects, the histology images are analyzed by a machine, e.g., a computer by way of machine learning, and the CD8 expression is characterized by the machine.

In some aspects, CD8 localization is measured using an immunostaining and imaging assay. In some aspects, the results of the assay are not analyzed by a human, e.g., a pathologist, and the CD8 expression is not characterized by the human. In some aspects, the results of the assay are analyzed by a machine, e.g., a computer by way of machine learning, and the CD8 expression is characterized by the machine.

In certain aspects, the CD8 localization is measured by immunostaining and imaging followed by classification of the CD8 localization in the tumor sample. CD8 localization classification can be conducted using any methods known in the art. In some aspects, CD8 localization classification is not performed by a human. In some aspects, CD8 localization classification is not performed by a pathologist. In some aspects, CD8 localization classification is performed by a computing device.

Some aspects of the present disclosure are directed to a method of identifying a subject suitable for a therapy comprising an anti-PD-⅟PD-L1 antagonist, comprising receiving, by at least one processor of a computing device, a plurality of histology images of tumor samples in a plurality of patients; performing, by the at least one processor, an image analysis of the plurality of histology images to obtain CD8+ T-cell abundance in the tumor parenchyma and stroma in each of the plurality of histology images; training, by the at least one processor, a machine learning algorithm using results of the image analysis and the CD8+ T-cell abundance in the tumor parenchyma and stroma; generating, by the at least one processor, a machine learning feature space comprising a plurality of classifications based on the training; and identifying, by the at least one processor, boundaries between the plurality of classifications in the machine learning feature space. In some aspects, performing the image analysis of the plurality of histology images comprises applying an artificial neural network to the plurality of histology images. In some aspects, the CD8+ T-cell abundance comprises a graphical representation of a relationship between percentages of the stromal CD8+ T-cells and percentages of the parenchymal CD8+ T-cells with respect to the total number of T-cells present in each of the plurality of histology images.

In some aspects, the method further comprises applying, by the at least one processor of the computing device, a polar coordinate transformation of the graphical representation, resulting in a polar plot; and using the polar plot to train the machine learning algorithm. In some aspects, the plurality of classifications comprises inflamed, desert, excluded, or balanced. In some aspects, the machine-learning algorithm comprises a random forest classifier algorithm. In some aspects, the method further comprises determining a classification for each of the plurality of histology images based on the machine learning feature space. In some aspects, the method further comprises validating results from the machine learning feature space by comparing a label for each of the plurality of histology images obtained by at least one pathologist to the classification for each of the plurality of histology images. In some aspects, the method further comprises receiving, by the at least one processor of the computing device, an additional histology image; performing an additional image analysis of the additional histology image and obtaining an additional CD8+ T-cell abundance in the tumor parenchyma and stroma in the additional histology image; applying the machine learning algorithm to results from the additional image analysis and the additional CD8+ T-cell abundance; and determining a classification for the additional histology image based on the machine learning feature space.

Other aspects of the present disclosure are directed to a system comprising: a memory; and a processor coupled to the memory, where the processor is configured to: receive a plurality of histology images of tumor samples in a plurality of patients; perform an image analysis of the plurality of histology images to obtain a CD8+ T-cell abundance in the tumor parenchyma and stroma in each of the plurality of histology images; train a machine learning algorithm using results of the image analysis and the CD8+ T-cell abundance in the tumor parenchyma and stroma; generate a machine learning feature space comprising a plurality of classifications based on the training; identify boundaries between the plurality of classifications in the machine learning feature space; and store the machine learning feature space and data regarding the boundaries in the memory. In some aspects, performing the image analysis of the plurality of histology images comprises applying an artificial neural network to the plurality of histology images, and wherein the machine-learning algorithm comprises a random forest classifier algorithm. In some aspects, the CD8+ T-cell abundance comprises a graphical representation of a relationship between percentages of the stromal CD8+ T-cells and percentages of the parenchymal CD8+ T-cells with respect to the total number of T-cells present in each of the plurality of histology images. In some aspects, the processor is further configured to: receive an additional histology image; perform an additional image analysis of the additional histology image and obtaining an additional CD8+ T-cell abundance in the tumor parenchyma and stroma in the additional histology image; apply the machine learning algorithm to results from the additional image analysis and the additional CD8+ T-cell abundance; and determine a classification for the additional histology image based on the machine learning feature space. In some aspects, the plurality of classifications comprises inflamed, desert, excluded, or balanced.

Other aspects of the present disclosure are directed to a non-transitory computer-readable medium having instructions stored thereon, execution of which, by one or more processors of a device, cause the one or more processors to perform operations comprising: receiving a plurality of histology images of tumor samples in a plurality of patients; performing an image analysis of the plurality of histology images to obtain a CD8+ T-cell abundance in the tumor parenchyma and stroma in each of the plurality of histology images; training a machine learning algorithm using results of the image analysis and the CD8+ T-cell abundance in the tumor parenchyma and stroma; generating a machine learning feature space comprising a plurality of classifications based on the training; and identifying boundaries between the plurality of classifications in the machine learning feature space. In some aspects, performing the image analysis of the plurality of histology images comprises applying an artificial neural network to the plurality of histology images. In some aspects, the machine-learning algorithm comprises a random forest classifier algorithm. In some aspects, the CD8+ T-cell abundance comprises a graphical representation of a relationship between percentages of the stromal CD8+ T-cells and percentages of the parenchymal CD8+ T-cells with respect to the total number of T-cells present in each of the plurality of histology images. In some aspects, the operations further comprising: receiving an additional histology image; performing an additional image analysis of the additional histology image and obtaining an additional CD8+ T-cell abundance in the tumor parenchyma and stroma in the additional histology image; applying the machine learning algorithm to results from the additional image analysis and the additional CD8+ T-cell abundance; and determining a classification for the additional histology image based on the machine learning feature space. In some aspects, the plurality of classifications comprises inflamed, desert, excluded, or balanced.

In other aspects, CD8 localization is measured by assaying for expression of one or more additional biomarkers. In some aspects, the expression profile of the one or more additional biomarkers indicates whether there is high CD8 localization in the tumor (e.g., inflamed CD8 localization phenotype) or in the stroma (e.g., excluded CD8 localization phenotype). In some aspects, CD8 localization is measured using a genome expression profiling (GEP) assay. Any method known in the art for measuring the expression of a particular gene or a panel of genes can be used in the methods of the present disclosure. In some aspects, the expression of one or more of the inflammatory genes in the inflammatory gene panel is determined by detecting the presence of mRNA transcribed from the inflammatory gene, the presence of a protein encoded by the inflammatory gene, or both.

In any of the methods comprising the measurement of CD8 in a test tissue sample, however, it should be understood that the step comprising the provision of a test tissue sample obtained from a patient is an optional step.

### II.A.2. PD-L1 Expression

In order to assess the PD-L1 expression, in some aspects, a test tissue sample can be obtained from the patient who is in need of the therapy. In another aspect, the assessment of PD-L1 expression can be achieved without obtaining a test tissue sample. In some aspects, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 in a test tissue sample, however, it should be understood that the step comprising the provision of a test tissue sample obtained from a patient is an optional step. It should also be understood that in certain aspects the “measuring” or “assessing” step to identify, or determine the number or proportion of, cells in the test tissue sample that express PD-L1 (e.g., the expression of PD-L1 on the cell surface) is performed by a transformative method of assaying for PD-L1 expression, for example by performing a reverse transcriptase-polymerase chain reaction (RT-PCR) assay or an IHC assay. In certain other aspects, no transformative step is involved and PD-L1 expression is assessed by, for example, reviewing a report of test results from a laboratory. In certain aspects, the steps of the methods up to, and including, assessing PD-L1 expression provides an intermediate result that may be provided to a physician or other healthcare provider for use in selecting a suitable candidate for the anti-PD-1 antibody or anti-PD-L1 antibody therapy. In certain aspects, the steps that provide the intermediate result is performed by a medical practitioner or someone acting under the direction of a medical practitioner. In other aspects, these steps are performed by an independent laboratory or by an independent person such as a laboratory technician.

In certain aspects of any of the present methods, the proportion of cells that express PD-L1 is assessed by performing an assay to determine the presence of PD-L1 RNA. In further aspects, the presence of PD-L1 RNA is determined by RT-PCR, in situ hybridization or RNase protection. In other aspects, the proportion of cells that express PD-L1 is assessed by performing an assay to determine the presence of PD-L1 polypeptide. In further aspects, the presence of PD-L1 polypeptide is determined by immunohistochemistry (IHC), enzyme-linked immunosorbent assay (ELISA), in vivo imaging, or flow cytometry. In some aspects, PD-L1 expression is assayed by IHC. In other aspects of all of these methods, cell surface expression of PD-L1 is assayed using, e.g., IHC or in vivo imaging.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM), and multiphoton microscopy (MPM) will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2(12):a003848 (2010)). Antibody specificity, coupled with the sensitivity and resolution of PET, makes immunoPET imaging particularly attractive for monitoring and assaying expression of antigens in tissue samples (McCabe and Wu, “Positive progress in immunoPET-not just a coincidence,” Cancer Biother. Radiopharm. 25(3):253-61 (2010); Olafsen et al., “ImmunoPET imaging of B-cell lymphoma using 124I-anti-CD20 scFv dimers (diabodies),” Protein Eng. Des. Sel. 23(4):243-9 (2010)). In certain aspects of any of the present methods, PD-L1 expression is assayed by immunoPET imaging. In certain aspects of any of the present methods, the proportion of cells in a test tissue sample that express PD-L1 is assessed by performing an assay to determine the presence of PD-L1 polypeptide on the surface of cells in the test tissue sample. In certain aspects, the test tissue sample is a FFPE tissue sample. In other aspects, the presence of PD-L1 polypeptide is determined by IHC assay. In further aspects, the IHC assay is performed using an automated process. In some aspects, the IHC assay is performed using an anti-PD-L1 monoclonal antibody to bind to the PD-L1 polypeptide.

In one aspect of the present methods, an automated IHC method is used to assay the expression of PD-L1 on the surface of cells in FFPE tissue specimens. In some aspects, the immunostained, e.g., IHC, images are further analyzed using a machine-learning algorithm. In some aspects, the immunostained, e.g., IHC, images are analyzed by a pathologist. This disclosure provides methods for detecting the presence of human PD-L1 antigen in a test tissue sample, or quantifying the level of human PD-L1 antigen or the proportion of cells in the sample that express the antigen, which methods comprise contacting the test sample, and a negative control sample, with a monoclonal antibody that specifically binds to human PD- L1, under conditions that allow for formation of a complex between the antibody or portion thereof and human PD-L1. In certain aspects, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In a particular aspect, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a postprimary blocking agent; incubating with NovoLink Polymer; adding a chromogen substrate and developing; and counterstaining with hematoxylin.

For assessing PD-L1 expression in tumor tissue samples, in some aspects, a pathologist examines the number of membrane PD-L1+ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one aspect, the threshold number of cells that needs to be PD-L1 positive is at least about 100, at least about 125, at least about 150, at least about 175, or at least about 200 cells. In certain aspects, the threshold number of cells that need to be PD-L1 positive is at least about 100 cells. In some aspects, the pathologist can be replaced using artificial intelligence.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain aspects of these scoring methods, the samples are scored by two pathologists operating independently, and the scores are subsequently consolidated. In certain other aspects, the identification of positive and negative cells is scored using appropriate software.

A histoscore (also described as H-score) is used as a more quantitative measure of the IHC data. The histoscore is calculated as follows:

$$\begin{matrix}\text{Histoscore} = \left[\left(\% \text{tumor x 1} \left(\text{low intensity}\right)\right) + \left(\% \text{tumor x}\right)\right) \\ \left(\left(\text{2} \left(\text{medium intensity}\right)\right) + \left(\% \text{tumor x 3} \left(\text{high intensity}\right)\right)\right]\end{matrix}$$

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 expression in a test tissue sample IHC is to determine the adjusted inflammation score (AIS) score defined as the density of inflammation multiplied by the percent PD-L1 expression by tumor-infiltrating inflammatory cells (Taube et al., “Colocalization of inflammatory response with B7-h1 expression in human melanocytic lesions supports an adaptive resistance mechanism of immune escape,” Sci. Transl. Med. 4(127):127ra37 (2012)).

### II.B. Methods of Treatment

Certain aspects of the present disclosure are directed to methods of identifying a subject suitable for a therapy and then administering the therapy to the suitable subject. The methods of identifying a suitable subject described herein can be used in advance of any immuno-oncology (I-O) therapy. In some aspects, the suitable subject is to be administered and/or subsequently administered an antibody or antigen-binding fragment thereof that specifically binds a protein selected from PD-1, PD-L1, CTLA-4, LAG-3, TIGIT, TIM3, CSF1R, NKG2a, OX40, ICOS, CD137, KIR, TGFβ, IL-10, IL-8, IL-2, CD96, VISTA, B7-H4, Fas ligand, CXCR4, mesothelin, CD27, GITR, MICA, MICB, and any combination thereof.

In some aspects, the suitable subject is to be administered and/or subsequently administered an anti-PD-⅟PD-L1 antagonist. In certain aspects, the anti-PD-⅟PD-L1 antagonist is an anti-PD-1 or an anti-PD-L1 antibody. In some aspects, the suitable subject is to be administered and/or subsequently administered an antibody or antigen-binding fragment thereof that specifically binds PD-1. In some aspects, the suitable subject is to be administered and/or subsequently administered an antibody or antigen-binding fragment thereof that specifically binds PD-L1.

In some aspects, the subject is to be further administered and/or subsequently further administered an anti-CTLA-4 agonist. In some aspects, the suitable subject is to be administered and/or subsequently administered an antibody or antigen-binding fragment thereof that specifically binds CTLA-4.

In some aspects, the suitable subject is to be administered and/or subsequently administered more than one antibody or antigen-binding fragment thereof disclosed herein. In some aspects, the suitable subject is to be administered and/or subsequently administered at least two antibodies or antigen-binding fragments thereof. In some aspects, the suitable subject is to be administered and/or subsequently administered at least three antibodies or antigen-binding fragments thereof. In certain aspects the suitable subject is to be administered and/or subsequently administered an antibody or antigen-binding fragment thereof that specifically binds PD-1 and an antibody or antigen-binding fragment thereof that specifically binds CTLA-4. In certain aspects the suitable subject is to be administered and/or subsequently administered an antibody or antigen-binding fragment thereof that specifically binds PD-L1 and an antibody or antigen-binding fragment thereof that specifically binds CTLA-4.

In certain aspects, the therapy is administered to the suitable subject after CD8 localization and PD-L1 expression has been assayed. In some aspects, the therapy is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days after CD8 localization and PD-L1 expression has been assayed.

Certain aspects of the present disclosure are directed to methods of treating a cancer in a human subject, comprising administering an anti-PD-⅟anti-PD-L1 antagonist to a subject, wherein the subject is identified as having a tumor exhibiting: (i) an excluded CD8 localization phenotype; and (ii) a negative PD-L1 expression status (“PD-L1 negative”). Some aspects of the present disclosure are directed to methods of identifying a subject suitable for

### II.C. Anti-PD-⅟PD-L1/CTLA-4 Antagonists

Certain aspects of the present disclosure are directed to methods of treating a suitable subject, as determined according to a method disclosed herein, using an anti-PD-⅟PD-L1 antagonist therapy. Some aspects of the present disclosure are directed to methods of treating a suitable subject, as determined according to a method disclosed herein, using an anti-PD-⅟PD-L1 antagonist and an anti-CTLA-4 antagonist therapy. Any anti-PD-⅟PD-L1/CTLA-4 antagonists known in the art can be used in the methods described herein. In some aspects, the anti-PD-1 antagonist comprises an anti-PD-1 antibody.

In some aspects, the subject is administered a single anti-PD-⅟PD-L1 antagonist, i.e., a monotherapy. In some aspects, the subject is administered an anti-PD-1 antibody monotherapy. In some aspects, the subject is administered an anti-PD-L1 antibody monotherapy. In some aspects, the subject is administered a combination therapy comprising a first anti-PD-⅟PD-L1 antagonist and an additional anticancer therapy. In some aspects, the additional anti-cancer agent comprises a second I-O therapy, a chemotherapy, a standard of care therapy, or any combination thereof.

In certain aspects, the subject is administered a combination therapy comprising an anti-PD-1 antibody and a second anti-cancer agent. In certain aspects, the subject is administered a combination therapy comprising an anti-PD-1 antibody and an anti-CTLA-4 antibody. In certain aspects, the subject is administered a combination therapy comprising an anti-PD-L1 antibody and an anti-CTLA-4 antibody.

### II.C.1. Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1 x 10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475 ; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHIPHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one aspect, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another aspect, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. No. 8,008,449 and 8,779,105; WO 2013/173223). In some aspects, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one aspect, the anti-PD-1 antibody is administered at a dose of about 5 mg/kg body weight about once every 3 weeks. In another aspect, the anti-PD-1 antibody, e.g., nivolumab, is administered at a dose of about 3 mg/kg body weight about once every 2 weeks. In other aspects, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some aspects, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one aspect, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another aspects, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some aspects, the anti-PD-1 antibody is administered as a flat dose of about 200 mg at about once every 3 weeks. In other aspects, the anti-PD-1 antibody is administered as a flat dose of about 200 mg at about once every 2 weeks. In other aspects, the anti-PD-1 antibody is administered as a flat dose of about 240 mg at about once every 2 weeks. In certain aspects, the anti-PD-1 antibody is administered as a flat dose of about 480 mg at about once every 4 weeks.

In some aspects, nivolumab is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 3 weeks. In some aspects, nivolumab is administered at a flat dose of about 480 mg once about every 4 weeks.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 4 weeks.

In some aspects, the PD-1 inhibitor is a small molecule. In some aspects, the PD-1 inhibitor comprises a millamolecule. In some aspects, the PD-1 inhibitor comprises a macrocyclic peptide. In certain aspects, the PD-1 inhibitor comprises BMS-986189. In some aspects, the PD-1 inhibitor comprises an inhibitor disclosed in International Publication No. WO2014/151634, which is incorporated by reference herein in its entirety. In some aspects, the PD-1 inhibitor comprises INCMGA00012 (Insight Pharmaceuticals). In some aspects, the PD-1 inhibitor comprises a combination of an anti-PD-1 antibody disclosed herein and a PD-1 small molecule inhibitor.

### II.C.2. Anti-PD-L1 Antibodies Useful for the Disclosure

In certain aspects, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in US Pat. No. 9,580,507. Anti-PD-L1 human monoclonal antibodies disclosed in U.S. Patent No. 9,580,507 have been demonstrated to exhibit one or more of the following characteristics: (a) bind to human PD-L1 with a K_{D} of 1 x 10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see US 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular aspect, the anti-PD-L1 antibody is durvalumab. In other aspects, the anti-PD-L1 antibody is avelumab. In some aspects, the anti-PD-L1 antibody is atezolizumab.

In some aspects, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some aspects, the anti-PD-L1 antibody is administered at a dose of about 15 mg/kg body weight at about once every 3 weeks. In other aspects, the anti-PD-L1 antibody is administered at a dose of about 10 mg/kg body weight at about once every 2 weeks.

In other aspects, the anti-PD-L1 antibody useful for the present disclosure is a flat dose. In some aspects, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some aspects, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some aspects, the anti-PD-L1 antibody is administered as a flat dose of about 1200 mg at about once every 3 weeks. In other aspects, the anti-PD-L1 antibody is administered as a flat dose of about 800 mg at about once every 2 weeks. In other aspects, the anti-PD-L1 antibody is administered as a flat dose of about 840 mg at about once every 2 weeks.

In some aspects, atezolizumab is administered as a flat dose of about 1200 mg once about every 3 weeks. In some aspects, atezolizumab is administered as a flat dose of about 800 mg once about every 2 weeks. In some aspects, atezolizumab is administered as a flat dose of about 840 mg once about every 2 weeks.

In some aspects, avelumab is administered as a flat dose of about 800 mg once about every 2 weeks.

In some aspects, durvalumab is administered at a dose of about 10 mg/kg once about every 2 weeks. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the PD-L1 inhibitor is a small molecule. In some aspects, the PD-L1 inhibitor comprises a millamolecule. In some aspects, the PD-L1 inhibitor comprises a macrocyclic peptide. In certain aspects, the PD-L1 inhibitor comprises BMS-986189.

In some aspects, the PD-L1 inhibitor comprises a millamolecule having a formula set forth in formula (I):

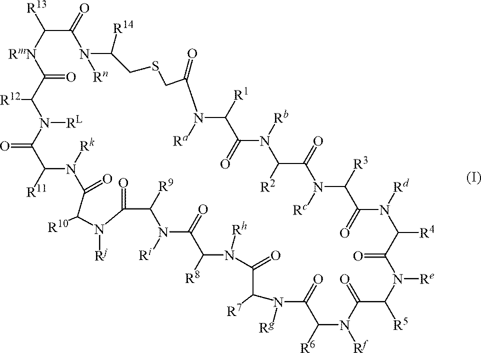

wherein R¹—R¹³ are amino acid side chains, R^{a}—Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In certain aspects the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety.

In some aspects, the PD-L1 inhibitor comprises a combination of an anti-PD-L1 antibody disclosed herein and a PD-L1 small molecule inhibitor disclosed herein.

### II.C.3. Anti-CTLA-4 Antibodies

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. Nos. 6,984,720 have been demonstrated to exhibit one or more of the following characteristics: (a) binds specifically to human CTLA-4 with a binding affinity reflected by an equilibrium association constant (Kα) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kα) of at least about 10³, about 10⁴, or about 10⁵ M⁻¹ S⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular aspects, the anti-CTLA-4 antibody is ipilimumab.

In particular aspects, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular aspects, the CTLA-4 antibody is tremelimumab.

In particular aspects, the CTLA-4 antibody is MK-1308.

In particular aspects, the CTLA-4 antibody is AGEN-1884.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain aspects, the anti-CTLA-4 antibody or antigen-binding portion thereof cross-competes with ipilimumab and/or tremelimumab for binding to human CTLA-4.

In some aspects, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some aspects, the anti-CTLA-4 antibody or antigen-binding portion thereof is administered at a dose of 1 mg/kg or 3 mg/kg body weight once every 3, 4, 5, or 6 weeks. In one aspect, the anti-CTLA-4 antibody or antigen-binding portion thereof is administered at a dose of 3 mg/kg body weight once every 2 weeks. In another aspect, the anti-PD-1 antibody or antigen-binding portion thereof is administered at a dose of 1 mg/kg body weight once every 6 weeks.

In some aspects, the anti-CTLA-4 antibody or antigen-binding portion thereof is administered as a flat dose. In some aspects, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 100 mg to about 1000 mg, from about 100 mg to about 900 mg, from about 100 mg to about 800 mg, from about 100 mg to about 700 mg, from about 100 mg to about 100 mg, from about 100 mg to about 500 mg, from about 100 mg to about 480 mg, or from about 240 mg to about 480 mg. In one aspect, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another aspect, the anti-CTLA-4 antibody or antigen-binding portion thereof is administered as a flat dose about once every 1, 2, 3, 4, 5, 6, 7, or 8 weeks.

In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses.

### II.D. Additional Anti-cancer Therapies

In some aspects of the present disclosure, the methods disclosed herein further comprise administering an anti-PD-⅟PD-L1 antagonist, e.g., an anti-PD-1 antibody or an anti-PD-L1 antibody, and one or more additional anti-cancer therapies. In certain aspects, the method comprising administering (i) a first anti-PD-⅟PD-L1 antagonist, e.g., an anti-PD-1 antibody or an anti-PD-L1 antibody), and (ii) one or more additional anti-cancer therapies. In certain aspects, the method comprising administering (i) a first anti-PD-⅟PD-L1 antagonist, e.g., an anti-PD-1 antibody or an anti-PD-L1 antibody), (ii) an anti-CTLA-4 antagonist, e.g., an anti-CTLA-4 antibody, and (iii) one or more additional anti-cancer therapies.

The additional anti-cancer therapy can comprise any therapy known in the art for the treatment of a tumor in a subject and/or any standard-of-care therapy, as disclosed herein. In some aspects, the additional anti-cancer therapy comprises a surgery, a radiation therapy, a chemotherapy, an immunotherapy, or any combination thereof. In some aspects, the additional anti-cancer therapy comprises a chemotherapy, including any chemotherapy disclosed herein.

Any chemotherapy known in the art can be used in the methods disclosed herein. In some aspects, the chemotherapy is a platinum based-chemotherapy. Platinum-based chemotherapies are coordination complexes of platinum. In some aspects, the platinum-based chemotherapy is a platinum-doublet chemotherapy. In some aspects, the chemotherapy is administered at the approved dose for the particular indication. In other aspects, the chemotherapy is administered at any dose disclosed herein. In some aspects, the platinum-based chemotherapy is cisplatin, carboplatin, oxaliplatin, satraplatin, picoplatin, Nedaplatin, Triplatin, Lipoplatin, or combinations thereof. In certain aspects, the platinum-based chemotherapy is any other platinum-based chemotherapy known in the art. In some aspects, the chemotherapy is the nucleotide analog gemcitabine. In an aspect, the chemotherapy is a folate antimetabolite. In an aspect, the folate antimetabolite is pemetrexed. In certain aspects the chemotherapy is a taxane. In other aspects, the taxane is paclitaxel. In some aspects, the chemotherapy is any other chemotherapy known in the art. In certain aspects, at least one, at least two or more chemotherapeutic agents are administered in combination with the I-O therapy. In some aspects, the I-O therapy is administered in combination with gemcitabine and cisplatin. In some aspects, the I-O therapy is administered in combination with pemetrexed and cisplatin. In certain aspects, the I-O therapy is administered in combination with gemcitabine and pemetrexed. In one aspect, the I-O therapy is administered in combination with paclitaxel and carboplatin. In an aspect, an I-O therapy is additionally administered.

In some aspects, the additional anti-cancer therapy comprises an immunotherapy (I-O therapy). In some aspects, the additional anti-cancer therapy comprises administration of an antibody or antigen-binding portion thereof that specifically binds LAG-3, TIGIT, TIM3, NKG2a, CSF1R, OX40, ICOS, MICA, MICB, CD137, KIR, TGFβ, IL-10, IL-8, B7-H4, Fas ligand, CXCR4, mesothelin, CD27, GITR, or any combination thereof.

### II.C.1. Anti-LAG-3 Antibodies

Anti-LAG-3 antibodies of the instant disclosure bind to human LAG-3. Antibodies that bind to LAG-3 have been disclosed in Int′l Publ. No. WO/2015/042246 and U.S. Publ. Nos. 2014/0093511 and 2011/0150892, each of which is incorporated by reference herein in its entirety.

An exemplary LAG-3 antibody useful in the present disclosure is 25F7 (described in U.S. Publ. No. 2011/0150892). An additional exemplary LAG-3 antibody useful in the present disclosure is BMS-986016. In one aspect, an anti-LAG-3 antibody useful for the composition cross-competes with 25F7 or BMS-986016. In another aspect, an anti-LAG-3 antibody useful for the composition binds to the same epitope as 25F7 or BMS-986016. In other aspects, an anti-LAG-3 antibody comprises six CDRs of 25F7 or BMS-986016. In another aspect, the anti-LAG-3 antibody is IMP731 (H5L7BW), MK-4280 (28G-10), REGN3767, humanized BAP050, IMP-701 (LAG-5250), TSR-033, BI754111, MGD013, or FS-118. These and other anti-LAG-3 antibodies useful in the claimed invention can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, and WO2014/140180, each of which is incorporated by reference herein in its entirety.

### II.C.2. Anti-CD137 Antibodies

Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669, each of which is incorporated by reference herein in its entirety.

In some aspects, the anti-CD137 antibody is urelumab (BMS-663513), described in U.S. Pat. No. 7,288,638 (20H4.9-IgG4 [10C7 or BMS-663513]). In some aspects, the anti-CD137 antibody is BMS-663031 (20H4.9-IgG1), described in U.S. Pat. No. 7,288,638. In some aspects, the anti-CD137 antibody is 4E9 or BMS-554271, described in U.S. Pat. No. 6,887,673. In some aspects, the anti-CD137 antibody is an antibody disclosed in U.S. Pat. Nos. 7,214,493; 6,303,121; 6,569,997; 6,905,685; or 6,355,476. In some aspects, the anti-CD137 antibody is 1D8 or BMS-469492; 3H3 or BMS-469497; or 3E1, described in U.S. Pat. No. 6,362,325. In some aspects, the anti-CD137 antibody is an antibody disclosed in issued U.S. Pat. No. 6,974,863 (such as 53A2). In some aspects, the anti-CD137 antibody is an antibody disclosed in issued U.S. Pat. No. 6,210,669 (such as 1D8, 3B8, or 3E1). In some aspects, the antibody is Pfizer’s PF-05082566 (PF-2566). In other aspects, an anti-CD137 antibody useful for the methods disclosed herein cross-competes with the anti-CD137 antibodies disclosed herein. In some aspects, an anti-CD137 antibody binds to the same epitope as the anti-CD137 antibody disclosed herein. In other aspects, an anti-CD137 antibody useful in the disclosure comprises six CDRs of the anti-CD137 antibodies disclosed herein.

### II.C.3. Anti-KIR Antibodies

Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int′l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448, each of which is incorporated by reference herein in its entirety.

One anti-KIR antibody useful in the present disclosure is lirilumab (also referred to as BMS-986015, IPH2102, or the S241P variant of 1-7F9), first described in Int′l Publ. No. WO 2008/084106. An additional anti-KIR antibody useful in the present disclosure is 1-7F9 (also referred to as IPH2101), described in Int′l Publ. No. WO 2006/003179. In one aspect, an anti-KIR antibody for the present composition cross competes for binding to KIR with lirilumab or I-7F9. In another aspect, an anti-KIR antibody binds to the same epitope as lirilumab or I-7F9. In other aspects, an anti-KIR antibody comprises six CDRs of lirilumab or I-7F9.

### II.C.4. Anti-GITR Antibodies

Anti-GITR antibodies useful in the methods disclosed herein include any anti-GITR antibody that binds specifically to human GITR target and activates the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int′l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841, each of which is incorporated by reference herein in its entirety.

In one aspect, an anti-GITR antibody useful in the present disclosure is TRX518 (described in, for example, Schaer et al. Curr Opin Immunol. (2012) Apr; 24(2): 217-224, and WO/2006/105021). In another aspect, the anti-GITR antibody is selected from MK4166, MK1248, and antibodies described in WO11/028683 and U.S. 8,709,424, and comprising, e.g., a VH chain comprising SEQ ID NO: 104 and a VL chain comprising SEQ ID NO: 105 (wherein the SEQ ID NOs are from WO11/028683 or U.S. 8,709,424). In certain aspects, an anti-GITR antibody is an anti-GITR antibody that is disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667. In certain aspects, an anti-GITR antibody is an anti-GITR antibody that is disclosed in WO2015/184099, e.g., antibody Hum231#1 or Hum231#2, or the CDRs thereof, or a derivative thereof (e.g., pab 1967, pab1975 or pab 1979). In certain aspects, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain aspects, an anti-GITR antibody is an anti-GITR antibody that is described in PCT/US2015/033991 (e.g., an antibody comprising the variable regions of 28F3, 18E10 or 19D3).

In certain aspects, the anti-GITR antibody cross-competes with an anti-GITR antibody described herein, e.g., TRX518, MK4166 or an antibody comprising a VH domain and a VL domain amino acid sequence described herein. In some aspects, the anti-GITR antibody binds the same epitope as that of an anti-GITR antibody described herein, e.g., TRX518 or MK4166. In certain aspects, the anti-GITR antibody comprises the six CDRs of TRX518 or MK4166.

### II.C.5. Anti-TIM3 Antibodies

Any anti-TIM3 antibody or antigen binding fragment thereof known in the art can be used in the methods described herein. In some aspects, the anti-TIM3 antibody is be selected from the anti-TIM3 antibodies disclosed in Int′l Publ. Nos.WO2018013818, WO/2015/117002 (e.g., MGB453, Novartis), WO/2016/161270 (e.g., TSR-022, Tesaro/AnaptysBio), WO2011155607, WO2016/144803 (e.g., STI-600, Sorrento Therapeutics), WO2016/071448, WO17055399; WO17055404, WO17178493, WO18036561, WO18039020 (e.g., Ly-3221367, Eli Lilly), WO2017205721, WO17079112; WO17079115; WO17079116, WO11159877, WO13006490, WO2016068802 WO2016068803, WO2016/111947, and WO/2017/031242, each of which is incorporated by reference herein in its entirety.

### II.C.6. Anti-OX40 Antibodies

Any antibody or antigen-binding fragment thereof that specifically binds OX40 (also known as CD134, TNFRSF4, ACT35 and/or TXGP1L) can be used in the methods disclosed herein. In some aspects, the anti-OX40 antibody is BMS-986178 (Bristol-Myers Squibb Company), described in Int′l Publ. No. WO20160196228. In some aspects, the anti-OX40 antibody is selected from the anti-OX40 antibodies described in Int′l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, and WO 17096182, each of which is incorporated by reference herein in its entirety.

### II.C.7. Anti-NKG2A Antibodies

Any antibody or antigen-binding fragment thereof that specifically binds NKG2A can be used in the methods disclosed herein. NKG2A is a member of the C-type lectin receptor family that is expressed on natural killer (NK) cells and a subset of T lymphocytes. Specifically, NKG2A primarily expressed on tumor infiltrating innate immune effector NK cells, as well as on some CD8+ T cells. Its natural ligand human leukocyte antigen E (HLA-E) is expressed on solid and hematologic tumors. NKG2A is an inhibitory receptor that blinds HLA-E.

In some aspects, the anti-NKG2A antibody may be BMS-986315, a human monoclonal antibody that blocks the interaction of NKG2A to its ligand HLA-E, thus allowing activation of an anti-tumor immune response. In some aspects, the anti-NKG2A antibody is a checkpoint inhibitor that activates T cells, NK cells, and/or tumor-infiltrating immune cells. In some aspects, the anti-NKG2A antibody is selected from the anti-NKG2A antibodies described in, for example, WO 2006/070286 (Innate Pharma S.A.; University of Genova); U.S. Pat. No. 8,993,319 (Innate Pharma S.A.; University of Genova); WO 2007/042573 (Innate Pharma S/A; Novo Nordisk A/S; University of Genova); U.S. Patent No. 9,447,185 (Innate Pharma S/A; Novo Nordisk A/S; University of Genova); WO 2008/009545 (Novo Nordisk A/S); US. Pat. Nos. 8,206,709; 8,901,283; 9,683,041 (Novo Nordisk A/S); WO 2009/092805 (Novo Nordisk A/S); U.S. Pat. Nos. 8,796,427 and 9,422,368 (Novo Nordisk A/S); WO 2016/134371 (Ohio State Innovation Foundation); WO 2016/032334 (Janssen); WO 2016/041947 (Innate); WO 2016/041945 (Academisch Ziekenhuis Leiden H.O.D.N. LUMC); WO 2016/041947 (Innate Pharma); and WO 2016/041945 (Innate Pharma), each of which is incorporated by reference herein in its entirety.

### II.C.8. Anti-ICOS Antibodies

Any antibody or antigen-binding fragment thereof that specifically binds ICOS can be used in the methods disclosed herein. ICOS is an immune checkpoint protein that is a member of the CD28-superfamily. ICOS is a 55-60 kDa type I transmembrane protein that is expressed on T cells after T cell activation and co-stimulates T-cell activation after binding its ligand, ICOS-L (B7H2). ICOS is also known as inducible T-cell co-stimulator, CVID1, AILIM, inducible costimulator, CD278, activation-inducible lymphocyte immunomediatory molecule, and CD278 antigen.

In some aspects, the anti-ICOS antibody is BMS-986226, a humanized IgG monoclonal antibody that binds to and stimulates human ICOS. In some aspects, the anti-ICOS antibody is selected from anti-ICOS antibodies described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), US. Pats. Nos. 7,045,615; 7,112,655, and 8,389,690 (Japan Tobacco Inc.), U.S. Pat. Nos. 9,738,718 and 9,771,424 (GlaxoSmithKline), and WO 2017/220988 (Kymab Limited), each of which is incorporated by reference herein in its entirety.

### II.C.9. Anti-TIGITAntibodies

Any antibody or antigen-binding fragment thereof that specifically binds TIGIT can be used in the methods disclosed herein. In some aspects, the anti-TIGIT antibody is BMS-986207. In some aspects, the anti-TIGIT antibody is clone 22G2, as described in WO 2016/106302. In some aspects, the anti-TIGIT antibody is MTIG7192A/RG6058/RO7092284, or clone 4.1D3, as described in WO 2017/053748. In some aspects, the anti-TIGIT antibody is selected from the anti-TIGIT antibodies described in, for example, WO 2016/106302 (Bristol-Myers Squibb Company) and WO 2017/053748 (Genentech).

### II.C.10. Anti-CSFIR Antibodies

Any antibody or antigen-binding fragment thereof that specifically binds CSF1R can be used in the methods disclosed herein. In some aspects, the anti-CSF1R antibody is an antibody species disclosed in any of international publications WO2013/132044, WO2009/026303, WO2011/140249, or WO2009/112245, such as cabiralizumab, RG7155 (emactuzumab), AMG820, SNDX 6352 (UCB 6352), CXIIG6, IMC-CS4, JNJ-40346527, MCS110, or the anti-CSF1R antibody in the methods is replaced with an anti-CSF1R inhibitor or anti-CSF1 inhibitor such as BLZ-945, pexidartinib (PLX3397, PLX108-01), AC-708, PLX-5622, PLX7486, ARRY-382, or PLX-73086.

### II.E. Tumors

In some aspects, the tumor is derived from a cancer selected from the group consisting of hepatocellular cancer, gastroesophageal cancer, melanoma, bladder cancer, lung cancer, kidney cancer, head and neck cancer, colon cancer, and any combination thereof. In certain aspects, the tumor is derived from a hepatocellular cancer, wherein the tumor has a high inflammatory signature score. In certain aspects, the tumor is derived from a gastroesophageal cancer, wherein the tumor has a high inflammatory signature score. In certain aspects, the tumor is derived from a melanoma, wherein the tumor has a high inflammatory signature score. In certain aspects, the tumor is derived from a bladder cancer, wherein the tumor has a high inflammatory signature score. In certain aspects, the tumor is derived from a lung cancer, wherein the tumor has a high inflammatory signature score. In certain aspects, the tumor is derived from a kidney cancer, wherein the tumor has a high inflammatory signature score. In certain aspects, the tumor is derived from a head and neck cancer, wherein the tumor has a high inflammatory signature score. In certain aspects, the tumor is derived from a colon cancer, wherein the tumor has a high inflammatory signature score.

In certain aspects, the subject has received one, two, three, four, five or more prior cancer treatments. In other aspects, the subject is treatment-naive. In some aspects, the subject has progressed on other cancer treatments. In certain aspects, the prior cancer treatment comprised an immunotherapy. In other aspects, the prior cancer treatment comprised a chemotherapy. In some aspects, the tumor has reoccurred. In some aspects, the tumor is metastatic. In other aspects, the tumor is not metastatic. In some aspects, the tumor is locally advanced.

In some aspects, the subject has received a prior therapy to treat the tumor and the tumor is relapsed or refractory. In certain aspects, the at least one prior therapy comprises a standard-of-care therapy. In some aspects, the at least one prior therapy comprises a surgery, a radiation therapy, a chemotherapy, an immunotherapy, or any combination thereof. In some aspects, the at least one prior therapy comprises a chemotherapy. In some aspects, the subject has received a prior immuno-oncology (I-O) therapy to treat the tumor and the tumor is relapsed or refractory. In some aspects, the subject has received more than one prior therapy to treat the tumor and the subject is relapsed or refractory. In other aspects, the subject has received either an anti-PD-1 or anti-PD-L1 antibody therapy.

In some aspects, the previous line of therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In one particular aspect, the platinum-based therapy comprises carboplatin.

In some aspects, the at least one prior therapy is selected from a therapy comprising administration of an anti-cancer agent selected from the group consisting of a platinum agent (e.g., cisplatin, carboplatin), a taxane agent (e.g., paclitaxel, albumin-bound paclitaxel, docetaxel), vinorelbine, vinblastine, etoposide, pemetrexed, gemcitabine, bevacizumab (AVASTIN®), erlotinib (TARCEVA®), crizotinib (XALKORI®), cetuximab (ERBITUX®), and any combination thereof. In certain aspects, the at least one prior therapy comprises a platinum-based doublet chemotherapy.

In some aspects, the subject has experienced disease progression after the at least one prior therapy. In certain aspects, the subject has received at least two prior therapies, at least three prior therapies, at least four prior therapies, or at least five prior therapies. In certain aspects, the subject has received at least two prior therapies. In one aspect, the subject has experienced disease progression after the at least two prior therapies. In certain aspects, the at least two prior therapies comprises a first prior therapy and a second prior therapy, wherein the subject has experienced disease progression after the first prior therapy and/or the second prior therapy, and wherein the first prior therapy comprises a surgery, a radiation therapy, a chemotherapy, an immunotherapy, or any combination thereof; and wherein the second prior therapy comprises a surgery, a radiation therapy, a chemotherapy, an immunotherapy, or any combination thereof. In some aspects, the first prior therapy comprises a platinum-based doublet chemotherapy, and the second prior therapy comprises a single-agent chemotherapy. In certain aspects, the single-agent chemotherapy comprises docetaxel.

### II.F. Pharmaceutical Compositions and Dosages

Therapeutic agents of the present disclosure can be constituted in a composition, e.g., a pharmaceutical composition containing an antibody and/or a cytokine and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing an antibody and/or a cytokine is suitable for non-parenteral, e.g., oral, administration. In some aspects, the subcutaneous injection is based on Halozyme Therapeutics’ ENHANZE® drug-delivery technology (see U.S. Pat. No. 7,767,429, which is incorporated by reference herein in its entirety). ENHANZE® uses a coformulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, antioxidant, aqueous and non-aqueous carriers, and/or adjuvants such as preservatives, wetting agents, emulsifying agents and dispersing agents. Therefore, in some aspects, the pharmaceutical composition for the present disclosure can further comprise recombinant human hyaluronidase enzyme, e.g., rHuPH20.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

Treatment is continued as long as clinical benefit is observed or until unacceptable toxicity or disease progression occurs. Nevertheless, in certain aspects, the antibodies disclosed herein are administered at doses that are significantly lower than the approved dosage, i.e., a subtherapeutic dosage, of the agent. The antibody can be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### III. Kits

Also within the scope of the present disclosure are kits comprising (a) an anti-PD-1 antibody or an anti-PD-L1 antibody for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage ranging from 0.1 to 10 mg/kg body weight of an anti-PD-1 antibody or a dosage ranging from 0.1 to 20 mg/kg body weight of an anti-PD-L1 antibody; and (b) instructions for using the anti-PD-1 antibody or the anti-PD-L1 antibody in the methods disclosed herein. This disclosure further provides a kit for treating a subject afflicted with a tumor, the kit comprising: (a) a dosage ranging from about 4 mg to about 500 mg of an anti-PD-1 antibody or a dosage ranging from about 4 mg to about 2000 mg of an anti-PD-L1 antibody; and (b) instructions for using the anti-PD-1 antibody or the anti-PD-L1 antibody in the methods disclosed herein. In some aspects, this disclosure provides a kit for treating a subject afflicted with a tumor, the kit comprising: (a) a dosage ranging from 200 mg to 800 mg of an anti-PD-1 antibody or a dosage ranging from 200 mg to 1800 mg of an anti-PD-L1 antibody; and (b) instructions for using the anti-PD-1 antibody or the anti-PD-L1 antibody in the methods disclosed herein.

In certain aspects for treating human patients, the kit comprises an anti-human PD-1 antibody disclosed herein, e.g., nivolumab or pembrolizumab. In certain aspects for treating human patients, the kit comprises an anti-human PD-L1 antibody disclosed herein, e.g., atezolizumab, durvalumab, or avelumab.

In some aspects, the kit further comprises an anti-CTLA-4 antibody. In certain aspects for treating human patients, the kit comprises an anti-human CTLA-4 antibody disclosed herein, e.g., ipilimumab, tremelimumab, MK-1308, or AGEN-1884.

In some aspects, the kit further includes a gene panel assay disclosed herein. In some aspects, the kit further includes instructions to administer the anti-PD-1 antibody or the anti-PD-L1 antibody to a suitable subject according to the methods disclosed herein.

All of the references cited above, as well as all references cited herein, are incorporated herein by reference in their entireties.

The following examples are offered by way of illustration and not by way of limitation.

### IV. Exemplary Embodiments of Artificial Intelligence and Machine Learning Assessment of Tumor Topology

Inflammation of the tumor microenvironment (TME), marked by infiltration of CD8+ T-cells, has been associated with improved clinical outcomes across multiple tumor types. Parenchymal infiltration of CD8+ T-cells has been associated with improved survival with immuno-oncology (I-O) treatment, and intratumoral localization also affects outcome, highlighting the importance of spatial analysis of CD8+ T-cells within the TME. CD8+ T-cell patterns within tumors, as assessed by immunostaining of histology images, are variable and may be classified as: (i) immune desert (minimal T-cell infiltrate); (ii) immune excluded (T-cells confined to tumor stroma or invasive margin); or (iii) Immune inflamed (T-cells infiltrating tumor parenchyma, positioned in proximity to tumor cells). Artificial intelligence (AI)-based image analysis can be used to characterize the tumor parenchymal and stromal compartments in the TME.

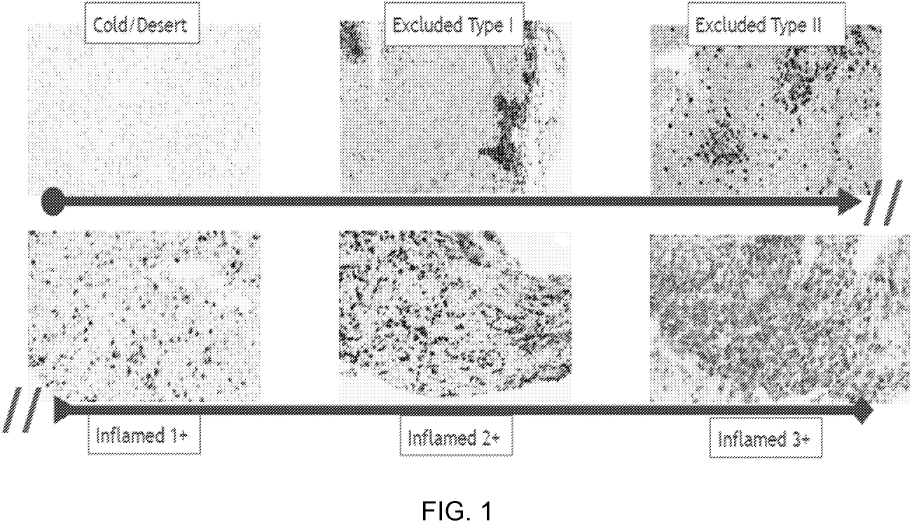

FIG. 1 illustrates example images of tumor tissue samples with various classifications using CD8+ histology images obtained by immunostaining, according to example embodiments. The tumor images show the various classifications of CD8+ T-cell patterns within the TME. The images in the top row in FIG. 1 show the immune desert and immune excluded classifications, and the images in the bottom row of FIG. 1 show the immune inflamed classification.

The immune desert classification indicates that the T-cells are minimal or absent from the TME. In some embodiments, the immune desert classification may be referred to herein as “desert” or “cold.” The immune excluded classification indicates that T-cells have accumulated in the tumor stroma without efficient infiltration of the tumor parenchyma. In some embodiments, the immune excluded classification may be referred to herein as “stromal.” The immune inflamed classification indicates that T-cells have infiltrated in the tumor parenchyma. In some embodiments, the immune inflamed classification may be referred to herein as “parenchymal.”

In some embodiments, there may be different levels within the immune excluded and immune inflamed classifications (e.g., first and second excluded levels, first, second, and third inflamed levels, and so forth) depending on the progression of the T-cells migrating within the TME. In some embodiments, a third inflamed level may indicate a higher number of T-cells infiltrating the parenchyma than the number of T-cell infiltrating the parenchyma in a first inflamed level. Although not shown in FIG. 1, there may be an intermediate classification between excluded and inflamed, referred to herein as “balanced.” The term “balanced” indicates an intermediate classification level between excluded and inflamed, in which there may be similar numbers of T-cells accumulated in the tumor stroma and T-cells accumulated in the tumor parenchyma.

In some embodiments, the tumor sample in the histology images obtained by immunostaining may be obtained by tissue biopsy and/or by resection of tumor tissue. In some embodiments, the tumor sample is a tumor tissue biopsy. In some embodiments, the tumor sample is a formalin-fixed, paraffin-embedded tumor tissue or a fresh-frozen tumor tissue. In some embodiments, the tumor sample is obtained from a stroma of the tumor. In some embodiments, the histology images obtained by immunostaining may be referred to herein as histology images.

In some embodiments, CD8 topology methods might not be standardized, resulting in inter-reviewer variability from different pathologists reviewing histology images. Interpretation of the CD8 topology from HISTOLOGY images may be confounded by various factors, such as different tumor types, limited tumor architecture due to biopsy or sampling, heterogeneity of inflammation within a tumor sample, and the like.

To address these problems in the field, embodiments described herein present a solution that provides a standardized, scalable approach using image analysis and machine learning techniques to facilitate review and assessment of CD8 topology of tumor tissue in patients.

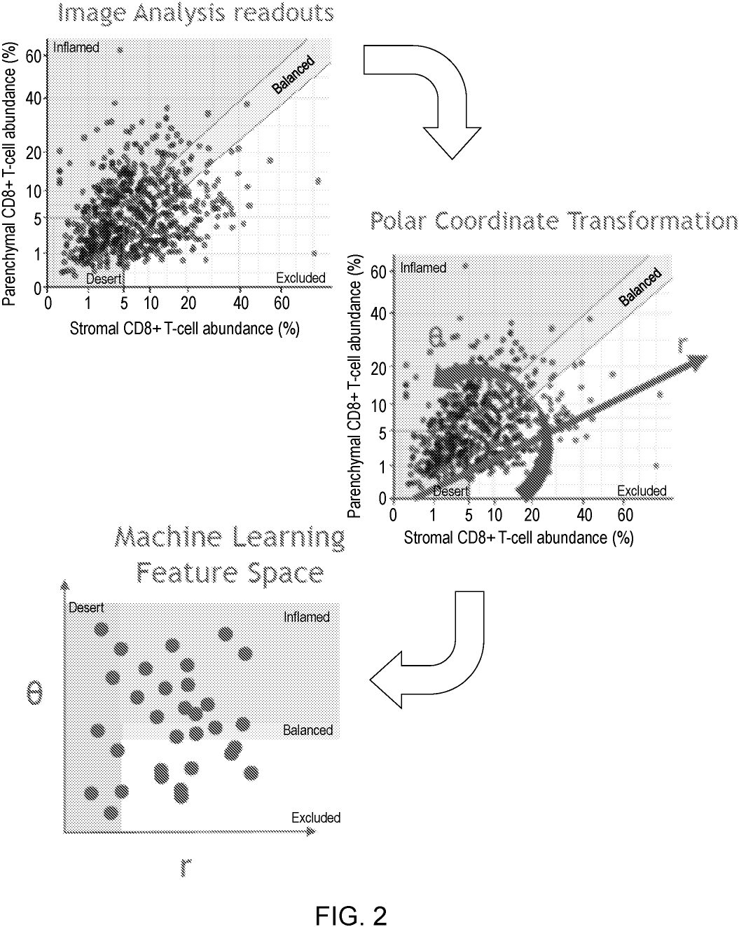

FIG. 2 is an example diagram illustrating a methodology for image analysis and machine learning based approaches for training a model for tumor topology classification, according to example embodiments. In particular, FIG. 2 shows three different stages of the methodology, including image analysis, polar coordinate transformation, and machine learning. The training data may include histology images obtained by immunostaining, which shows CD8+ T-cell patterns within a TME for a plurality of patients. These training images may have been labelled by trained topologists as classified into various categories. In some embodiments, the classification categories are “desert,” “excluded,” and “stromal.” In some embodiments, the classification categories include “balanced.”

In the first stage, the training data is processed to extract information from each histology image. In some embodiments, an image analysis process identifies and outputs a variety of parameters for each image. In some embodiments, the image parameters are already known, and the image analysis process selects a subset of parameters for further analysis. Such parameters may include, for example, the number of stromal CD8+ T-cells, the number of parenchymal CD8+ T-cells, and the number of all CD8+ T-cells in each image. Other parameters may include the density of stromal CD8+ T-cells and the density of parenchymal CD8+ T-cells in each image, which may be particularly useful if the total number of all CD8+ T-cells is not known or cannot be determined.

In some embodiments, the image analysis may obtain a CD8+ T-cell abundance in the tumor parenchyma and stroma in each histology image. In some embodiments, the CD8+ T-cell abundance may comprise a graphical representation of a relationship between percentages of the stromal CD8+ T-cells and percentages of the parenchymal CD8+ T-cells with respect to the total number of T-cells present in each of the plurality of histology images, as shown by the “image analysis readout” plot of FIG. 2. In some embodiments, the graphical representation may show density, percentage, and/or quantity of stromal CD8+ T-cells and parenchymal CD8+ T-cells in each image. In some embodiments, the image analysis may comprise any image recognition, processing, and/or analysis algorithm(s). In some embodiments, the image analysis may be performed by applying an artificial neural network (e.g., a convolutional neural network) to the plurality of histology images.

In the second stage, a polar coordinate transformation may be performed on the results from the image analysis to transform the image analysis readout graph into a polar plot with polar coordinates. In some embodiments, the polar coordinate transformation may comprise a mathematical transformation of the features derived during image analysis to a polar coordinate feature space.

In the third stage, a machine learning algorithm may be trained using the transformed results of the image analysis and the CD8+ T-cell abundance in the tumor parenchyma and stroma. In some embodiments, the polar coordinate transformation is skipped, such that the machine learning algorithm is trained using the results of the image analysis process without polar transformation. In some embodiments, the machine learning algorithm may comprise any type of classification algorithm, such as, e.g., a random forest classifier. In some embodiments, a machine learning algorithm may be trained using the same training data used to train the image analysis algorithm. In some embodiments, a random forest classifier may be trained using engineered features (e.g., image analysis derived features) and pathologist defined CD8+ topology. In some embodiments, labeled histology images (e.g., histology images that have been previously labeled with a classification by at least one pathologist) may be used to train the random forest classifier to provide classifications for additional histology images received. In some embodiments, the classifications include inflamed, desert, excluded, or balanced. In some embodiments, the machine learning algorithm may be referred to as a predictive model that is trained to predict classifications in histology images of tumors. In some embodiments, a recommendation for immunotherapy or treatment for a patient’s tumor may be generated based on determining a classification for at least one histology image of the patient’s tumor using the trained machine learning algorithm.

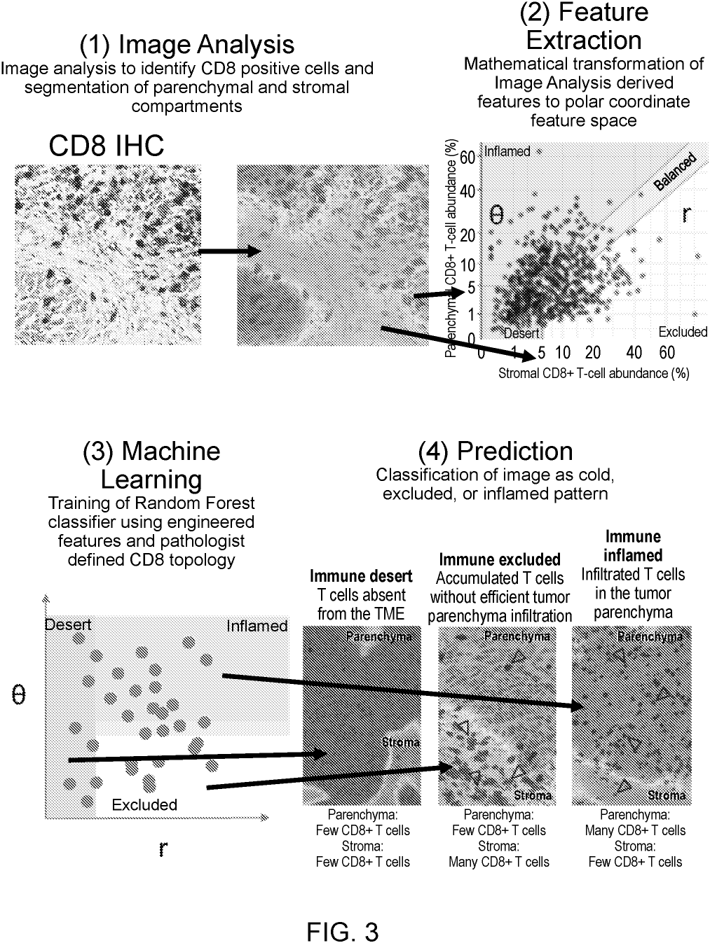

FIG. 3 is another example diagram illustrating the methodology for classification of tumor topology using image analysis and machine learning-based approaches, according to example embodiments. In some embodiments, FIG. 3 illustrates additional details for an embodiment of the methodology shown in FIG. 2. FIG. 3 illustrates four stages for training one or more machine learning algorithms for tumor topology classification and classifying new images using the trained algorithm, in which the stages include image analysis, feature extraction, machine learning, and prediction.

First, as shown in FIG. 3-(1), image analysis may be performed to identify CD8 positive cells and segmentation of parenchymal and stromal compartments in histology images of tumors. In some embodiments, the image analysis may include applying a neural network (e.g., a convolutional neural network) to a plurality of histology images to assess CD8+ T-cells in different parts of the tumor (e.g., tumor epithelium, stroma, and parenchyma) in each image. The image analysis tool may result in identifying values for a plurality of different parameters for each of the images in the plurality of histology images. In some embodiments, two parameters (e.g., number of stromal CD8+ T-cells and number of parenchymal CD8+ T-cells) may be selected for further analysis. In some embodiments, a CD8+ T-cell abundance in the tumor parenchyma and stroma for the plurality of histology images may be obtained from the image analysis.

Next, as shown in FIG. 3-(2), a feature extraction may be conducted by applying a mathematical transformation of image analysis-derived features to transform the data into a polar coordinate feature space. In some embodiments, the feature extraction may be a part of the image analysis process to identify the relationship between stromal CD8+ T-cells and parenchymal CD8+ T-cells.

After the mathematical transformation, as shown in FIG. 3-(3), a machine learning algorithm (e.g., a random forest classifier) may be trained using the engineered features and pathologist-defined CD8 topology. In some embodiments, training the machine learning algorithm may include generating a machine learning feature space comprising the plurality of classifications (e.g., inflamed, desert, excluded, or balanced). The machine learning algorithm may also be able to identify boundaries between the plurality of classifications in the machine learning feature space.

Once the machine learning algorithm has been trained, as shown in FIG. 3-(4), trained machine learning algorithm may classify the CD8 topology in new histology images as inflamed, desert, excluded, or balanced. Such a classification for a given patient’s image may then be used to diagnose a patient’s condition, determine an immune response of the patient, and/or be utilized to recommend or rule out treatment options for that patient.

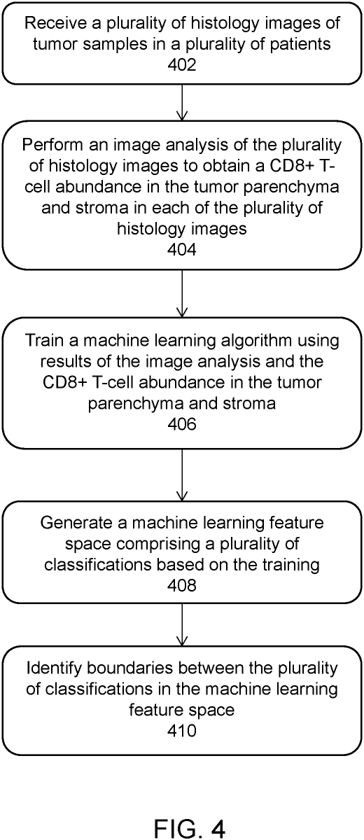

FIG. 4 is a flowchart illustrating a process for training a machine learning algorithm for classification of CD8 tumor topology, according to example embodiments. Method 400 may be performed by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously or in a different order than shown in FIG. 4, as will be understood by a person of ordinary skill in the art.

In operation 402, a plurality of histology images of tumor samples in a plurality of patients may be received by at least one processor of a computing device. In some embodiments, the histology images may comprise tumor tissue samples obtained using CD8+ immunostaining techniques and showing CD8+ T-cell patterns within the TME for a plurality of patients.

In operation 404, an image analysis of the plurality of histology images may be performed to obtain a CD8+ T-cell abundance in the tumor parenchyma and stroma in each of the plurality of histology images. In some embodiments, performing the image analysis of the plurality of histology images includes applying an artificial neural network (e.g., a convolutional neural network) to the plurality of histology images. In some embodiments, the CD8+ T-cell abundance in the tumor parenchyma and stroma may comprise a graphical representation of a relationship between percentages of the stromal CD8+ T-cells and percentages of the parenchymal CD8+ T-cells with respect to the total number of T-cells present in each of the plurality of histology images.

In operation 406, a machine learning algorithm may be trained using results of the image analysis and the CD8+ T-cell abundance in the tumor parenchyma and stroma. In some embodiments, a polar coordinate transformation may be applied to the graphical representation of the relationship between the stromal CD8+ T-cells and parenchymal CD8+ T-cells, and the resulting polar plot may be used to train the machine learning algorithm. In some embodiments, the machine learning algorithm comprises a random forest classifier algorithm.

In operation 408, a machine learning feature space comprising a plurality of classifications may be generated based on the training. In some embodiments, the plurality of classifications comprises inflamed, desert, excluded, or balanced.

In operation 410, boundaries between the plurality of classifications in the machine learning feature space may be identified. In some embodiments, the machine learning feature space and data regarding the boundaries between the plurality of classifications in the machine learning feature space may be stored in the memory of the computing device or computer system.

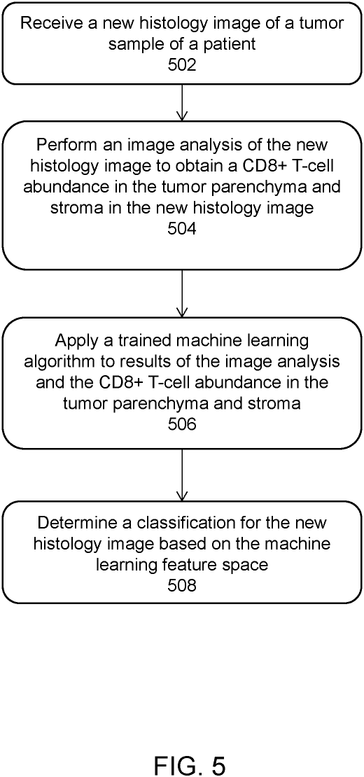

FIG. 5 is a flowchart illustrating the process for classifying CD8 tumor topology of a histology image using the trained machine learning algorithm, according to example embodiments. Method 500 may be performed by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously or in a different order than shown in FIG. 5, as will be understood by a person of ordinary skill in the art.

In operation 502, a new histology image of a tumor sample of a patient may be received by at least one processor of a computing device. In some embodiments, the new histology image may comprise a tumor tissue sample obtained using CD8+ immunostaining techniques and showing CD8+ T-cell patterns within the TME.

In operation 504, an image analysis of the new histology image may be performed to obtain a CD8+ T-cell abundance in the tumor parenchyma and stroma in the new histology image. This image analysis may be performed, for example, by the same image analysis algorithm(s) of operation 404 in FIG. 4.

In operation 506, a trained machine learning algorithm may be applied to results of the image analysis and the c CD8+ T-cell abundance in the tumor parenchyma and stroma. In some embodiments, the trained machine learning algorithm may be generated by method 400 in FIG. 4. In some embodiments, the trained machine learning algorithm may include a machine learning feature space that includes the different classifications for the CD8 topology (e.g., inflamed, desert, excluded, or balanced).

In operation 508, a classification for the new histology image may be determined using the machine learning feature space. In some embodiments, the machine learning algorithm may be able to determine where the patterns of stromal CD8+ T-cells and parenchymal CD8+ T-cells in the new histology image fall within the boundaries for the plurality of classifications in the machine learning feature space. Based on this mapping, the machine learning algorithm may output a classification for the new histology image.

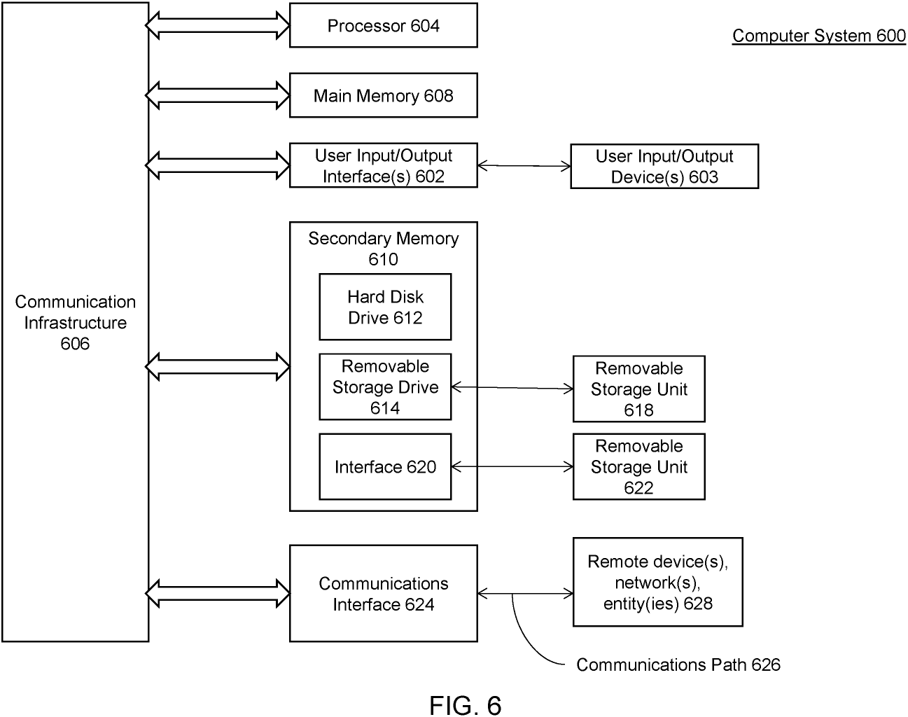

FIG. 6 is a block diagram of example components of computer system 600. One or more computer systems 600 may be used, for example, to implement any of the embodiments discussed herein, as well as combinations and sub-combinations thereof. In some embodiments, one or more computer systems 600 may be used to implement the methods 400 and 500 shown in FIGS. 4 and 5, respectively. Computer system 600 may include one or more processors (also called central processing units, or CPUs), such as a processor 604. Processor 604 may be connected to a communication infrastructure or bus 606.

Computer system 600 may also include user input/output interface(s) 602, such as monitors, keyboards, pointing devices, etc., which may communicate with communication infrastructure 606 through user input/output interface(s) 603

One or more of processors 604 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 600 may also include a main or primary memory 608, such as random access memory (RAM). Main memory 608 may include one or more levels of cache. Main memory 608 may have stored therein control logic (i.e., computer software) and/or data.

Computer system 600 may also include one or more secondary storage devices or memory 610. Secondary memory 610 may include, for example, a hard disk drive 612 and/or a removable storage drive 614.

Removable storage drive 614 may interact with a removable storage unit 618. Removable storage unit 618 may include a computer usable or readable storage device having stored thereon computer software (control logic) and/or data. Removable storage unit 618 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 614 may read from and/or write to removable storage unit 618.

Secondary memory 610 may include other means, devices, components, instrumentalities or other approaches for allowing computer programs and/or other instructions and/or data to be accessed by computer system 600. Such means, devices, components, instrumentalities or other approaches may include, for example, a removable storage unit 622 and an interface 620. Examples of the removable storage unit 622 and the interface 620 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 600 may further include a communication or network interface 624. Communication interface 624 may enable computer system 600 to communicate and interact with any combination of external devices, external networks, external entities, etc. (individually and collectively referenced by reference number 628). For example, communication interface 624 may allow computer system 600 to communicate with external or remote devices 628 over communications path 626, which may be wired and/or wireless (or a combination thereof), and which may include any combination of LANs, WANs, the Internet, etc. Control logic and/or data may be transmitted to and from computer system 600 via communication path 626.

Computer system 600 may also be any of a personal digital assistant (PDA), desktop workstation, laptop or notebook computer, netbook, tablet, smartphone, smartwatch or other wearables, appliance, part of the Internet-of-Things, and/or embedded system, to name a few nonlimiting examples, or any combination thereof.

Computer system 600 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 600 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 600, main memory 608, secondary memory 610, and removable storage units 618 and 622, as well as tangible articles of manufacture embodying any combination of the foregoing. Such control logic, when executed by one or more data processing devices (such as computer system 600), may cause such data processing devices to operate as described herein.

References in the Detailed Description to “one exemplary embodiment,” “an exemplary embodiment,” “an example exemplary embodiment,” etc., indicate that the exemplary embodiment described may include a particular feature, structure, or characteristic, but every exemplary embodiment might not necessarily include the particular feature, structure, or characteristic. Moreover, such phrases are not necessarily referring to the same exemplary embodiment. Further, when a particular feature, structure, or characteristic is described in connection with an exemplary embodiment, it is within the knowledge of those skilled in the relevant art(s) to affect such feature, structure, or characteristic in connection with other exemplary embodiments whether or not explicitly described.

The exemplary embodiments described herein are provided for illustrative purposes, and are not limiting. Other exemplary embodiments are possible, and modifications may be made to the exemplary embodiments within the spirit and scope of the disclosure. Therefore, the Detailed Description is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a general purpose computer, as described above.

The exemplary embodiments of artificial intelligence and machine-learning described herein for use in identifying CD8 topology can be applied to measure the expression of any biomarker known in the art, including any tumor biomarker. In some aspects, the tumor biomarker that is analyzed and/or characterized using the methods disclosed herein include, but are not limited to, PD-L1, PD-1, LAG3, CLTA-4, TIGIT, TIM3, NKG2a, CSF1R, OX40, ICOS, MICA, MICB, CD137, KIR, TGFβ, IL-10, IL-8, B7-H4, Fas ligand, CXCR4, mesothelin, CD27, GITR, and any combination thereof. The markers may also include morphologically identified markers without a staining antibody, such as lymphocytes, fibroblasts, macrophages, neutrophils, eosinophils, or any combination thereof. Similarly, although the examples herein are described in the context of tumors, the machine-learning based methods described herein may also be applicable for other tissue types in a variety of therapeutic uses, such as in fibrosis, cardiological, gastrointestinal, and other oncologic and non-oncologic therapeutic areas.

### EXAMPLES

### Example 1

Random forest AI-classifiers were trained to predict pathologist-assigned inflamed, excluded, and cold patterns on CD8-immunostaining using parenchymal and stromal CD8 measurements from a deep learning platform. Independently, AI-defined CD8-topology was compared with survival in retrospective analyses of all marker-evaluable, clinical baseline CD8-immunostaining in CA209-067 melanoma (MEL-NIVO+IPI arm, n=102); (MEL-NIVO arm, n=107) and CA209-275 urothelial carcinoma (UC-NIVO, n=263).

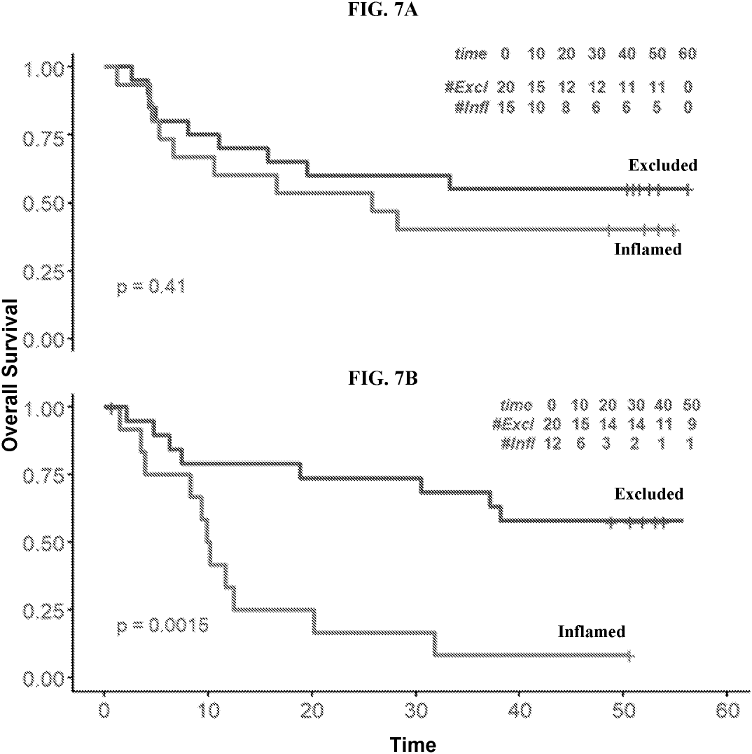

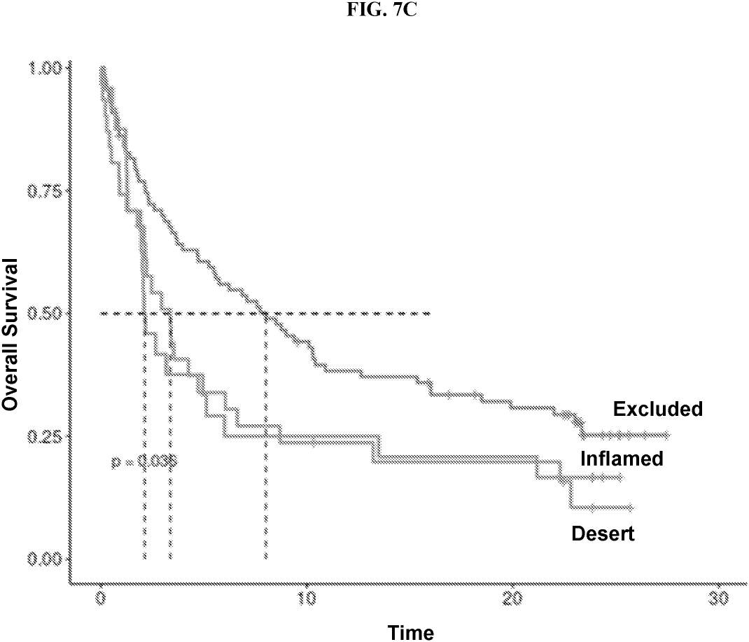

The PD-L1<1%/CD8-Excluded subset exhibited longer median overall survival (mOS) and lower hazard ratios (HR) compared to the PD-L1<1%/CD8-Inflamed population for all trial arms: [MEL-NIVO+IPI: mOS>50-months (n=20) versus 10.1-months (n=12), HR=0.23(95%CI:0.09-0.61); MEL-NIVO: mOS>50-months (n=20) versus 25.8-months (n=15), HR=0.68(95%CI:0.27-1.7); UC-NIVO: mOS=9.0-months (n=87) versus 3.1-months (n=24), HR=0.62(95%CI:0.38-1.00)] (FIGS. 7A-7C).

CD8-Excluded pattern demonstrated superior survival over CD8-Inflamed in the setting of PD-L1 negative tumors, and a composite-immunostaining approach combining CD8-topology with PD-L1 could yield improved patient selection across multiple tumor indications and treatment settings. Further studies are underway to identify mechanisms underlying these findings.

### Example 2

As described in Example 1, a random forest classifier was trained to predict CD8 topology using parenchymal and stromal CD8+ immune-cell measurements derived from a deeplearning platform. For model validation, pathologists manually classified CD8 immunohistochemistry in melanoma samples into inflamed (CD8+ cells in tumor parenchyma), excluded (CD8+ cells restricted to stroma), and desert (deficient in CD8+ cells) patterns. The association with overall survival (OS) was explored in a subset of patients with previously untreated metastatic melanoma who received nivolumab + ipilimumab (NIVO+IPI, n=102) or NIVO alone (n=107) in a phase 3 clinical trial. Retrospective analysis of baseline AI-defined CD8 topology was performed alone and combined with manually scored programmed death ligand 1 (PD-L1) expression on tumor cells.

Classifier model predictions were concordant with manual scoring (determined by a consensus of pathologists) and non-inferior to the agreement between 2 pathologists, via Cohen’s kappa coefficient k=0.79 and k=0.65, respectively. No statistically meaningful differences in outcomes were observed between CD8-excluded and CD8-inflamed phenotypes within the PD-L1 ≥1% population. However, patients with PD-L1 <1%/CD8-excluded tumors exhibited longer median OS compared with those with PD-L1 <1%/CD8-inflamed (Table 1). 38% (40/104) of PD-L1 <1% tumors were CD8-excluded. Within PD-L1 <1%, patients with an excluded phenotype also exhibited lower frequency of severe adverse events (grade ≥3) than patients with inflamed phenotype following treatment: NIVO+IPI, 75% (n=20) vs 91% (n=11); NIVO, 61% (n=18) vs 80% (n=15). Compared with PD-L1 status, the composite biomarker (AI-classified CD8-excluded plus PD-L1 ≥1%) identified a larger group of patients who had greater survival benefit with NIVO+IPI or NIVO alone (Table 2).

TABLE 1

Immunotherapy outcomes by CD8+ topology in PD-L1<1% melanoma

| | | | | |
|---|---|---|---|---|
| Treatment arm | NIVO+IPI | | NIVO | |
| Phenotype (n) | PD-L1 <1%, CD8-excluded (20) | PD-L1 <1%, CD8-inflamed (12) | PD-L1 <1%, CD8-excluded (20) | PD-L1 <1%, CD8-inflamed (15) |
| Median OS (months) | >50 | 10.1 | >50 | 25.8 |
| OS HR (95% Cl) | 0.23 (0.09-0.61), P<0.01 | | 0.68 (0.27-1.70), P=0.41 | |

TABLE 2

Composite biomarker outcomes in the study

| Treatment arm (n) | PD-L1 ≥1% | | Composite biomarker (PD-L1 ≥1% plus CD8-excluded) | |
|---|---|---|---|---|
| | n (%) | OS HR (95% Cl) | n (%) | OS HR (95% Cl) |
| NIVO+IPI (102) | 52 (51%) | 0.50 (0.29-0.89), P=0.017 | 72 (71%) | 0.35 (0.20-0.61), P<0.001 |
| NIVO (107) | 53 (50%) | 0.46 (0.27-0.79), P=0.005 | 73 (68%) | 0.37 (0.22-0.62), P<0.001 |

Hazard ratios represent patients with a PD-L1 expression of ≥1% compared with PD-L1 <1% or patients with a PD-L1 expression of ≥1% and CD8-excluded phenotype compared with PD-L1 expression <1% and not CD8-excluded.

This study combines AI-powered CD8 topology classifications with PD-L1 expression as a composite biomarker associated with immunotherapy response. In patients with PD-L1 <1% melanoma, median OS with NIVO+IPI was significantly longer in patients with CD8-excluded tumors than with an inflamed phenotype.

What is claimed is:

1. A pharmaceutical composition comprising an anti-PD-⅟PD-L1 antagonist for use in a method of treating a human subject afflicted with a tumor, wherein a tumor sample obtained from the subject exhibits:
(i) an excluded CD8 localization phenotype, and
(ii) a negative PD-L1 expression status.

2. The pharmaceutical composition for use of claim 1, wherein the subject is to be administered an anti-PD-⅟PD-L1 antagonist in combination with an anti-cancer agent.

3. The pharmaceutical composition for use of claim 1 or 2, wherein the subject is to be administered an anti-PD-⅟PD-L1 antagonist in combination with an anti-CTLA-4 antagonist.

4. The pharmaceutical composition for use of any one of claims 1 to 3, wherein the tumor sample is a tumor tissue biopsy.

5. The pharmaceutical composition for use of any one of claims 1 to 4, wherein the tumor sample is a formalin-fixed, paraffin-embedded tumor tissue or a fresh-frozen tumor tissue.

6. The pharmaceutical composition for use of any one of claims 1 to 5, wherein the CD8 localization is measured by staining the tumor sample with an antibody or an antigen-binding portion thereof that binds CD8.

7. The pharmaceutical composition of claim 6, wherein the tumor sample is imaged following the staining with the antibody.

8. The pharmaceutical composition for use of any one of claims 1 to 6, wherein the PD-L1 expression is measured by staining the tumor sample with an antibody or an antigen-binding portion thereof that specifically binds PD-L1.

9. The pharmaceutical composition for use of any one of claims 1 to 8, wherein the negative PD-L1 expression status is characterized by a tumor sample wherein less than about 1% of tumor cells express PD-L1.

10. The pharmaceutical composition for use of claim 7, wherein the PD-L1 expression is measured using an IHC assay.

11. The pharmaceutical composition for use of claim 10, wherein the IHC assay comprises an automated IHC assay.

12. The pharmaceutical composition for use of any one of claims 1 to 11, wherein the CD8 localization is measured by IHC followed by classification of the CD8 localization in the tumor sample.

13. The pharmaceutical composition for use of claim 12, wherein the classification is performed by a method comprising:
receiving, by at least one processor of a computing device, a plurality of histology images of tumor samples in a plurality of patients;
performing, by the at least one processor, an image analysis of the plurality of histology images to obtain a CD8+ T-cell abundance in the tumor parenchyma and stroma in each of the plurality of histology images;
training, by the at least one processor, a machine learning algorithm using results of the image analysis and the CD8+ T-cell abundance in the tumor parenchyma and stroma;
generating, by the at least one processor, a machine learning feature space comprising a plurality of classifications based on the training; and
identifying, by the at least one processor, boundaries between the plurality of classifications in the machine learning feature space.

14. A pharmaceutical composition comprising an anti-PD-⅟PD-L1 antagonist for use in a method of identifying a human subject suitable for an anti-PD-⅟PD-L1 antagonist therapy, wherein the method comprises (i) measuring an expression of PD-L1 in a tumor sample obtained from the subject, and (ii) measuring CD8 localization in the tumor sample;
wherein the CD8 localization is measured by staining the tumor sample with an antibody or an antigen-binding portion thereof that binds CD8, and classification of the CD8 localization in the tumor sample;
wherein the classification is performed by a method comprising:
receiving, by at least one processor of a computing device, a plurality of histology images of tumor samples in a plurality of patients;
performing, by the at least one processor, an image analysis of the plurality of histology images to obtain a CD8+ T-cell abundance in the tumor parenchyma and stroma in each of the plurality of histology images;
training, by the at least one processor, a machine learning algorithm using results of the image analysis and the CD8+ T-cell abundance in the tumor parenchyma and stroma;
generating, by the at least one processor, a machine learning feature space comprising a plurality of classifications based on the training; and
identifying, by the at least one processor, boundaries between the plurality of classifications in the machine learning feature space.

15. The pharmaceutical composition for use of claim 13 or 14, wherein performing the image analysis of the plurality of histology images comprises applying an artificial neural network to the plurality of histology images.

16. The pharmaceutical composition for use of claim 15, wherein the machine-learning algorithm comprises a random forest classifier algorithm.

17. The pharmaceutical composition for use any one of claims 13 to 16, wherein the CD8+ T-cell abundance comprises a graphical representation of a relationship between percentages of the stromal CD8+ T-cells and percentages of the parenchymal CD8+ T-cells with respect to the total number of T-cells present in each of the plurality of histology images.

18. The pharmaceutical composition for use of claim 17, further comprising: applying, by the at least one processor of the computing device, a polar coordinate transformation of the graphical representation, resulting in a polar plot; and using the polar plot to train the machine learning algorithm.

19. The pharmaceutical composition for use of any one of claims 13 to 18, wherein the plurality of classifications comprises inflamed, desert, excluded, or balanced.

20. The pharmaceutical composition for use of any one of claims 13 to 19, further comprising determining a classification for each of the plurality of histology images based on the machine learning feature space.

21. The pharmaceutical composition for use of claim 20, further comprising validating results from the machine learning feature space by comparing a label for each of the plurality of histology images obtained by at least one pathologist to the classification for each of the plurality of histology images.

22. The pharmaceutical composition for use of any one of claims 13 to 21, further comprising: receiving, by the at least one processor of the computing device, an additional histology image; performing an additional image analysis of the additional histology image and obtaining an additional CD8+ T-cell abundance in the tumor parenchyma and stroma in the additional histology image; applying the machine learning algorithm to results from the additional image analysis and the additional CD8+ T-cell abundance; and determining a classification for the additional histology image based on the machine learning feature space.

23. The pharmaceutical composition for use of any one of claims 1 to 22, wherein the CD8 localization is measured by measuring expression of a panel of genes in a tumor sample obtained from the subject.

24. The pharmaceutical composition for use of any one of claims 1 to 23, wherein a subject identified as having an excluded CD8 localization phenotype and a PD-L1 negative tumor is to be administered therapy comprising the anti-PD-⅟PD-L1 antagonist.

25. The pharmaceutical composition for use of any one of claims 1 to 23, wherein a subject identified as having an excluded CD8 localization phenotype and a PD-L1 negative tumor is to be administered therapy comprising the anti-PD-⅟PD-L1 antagonist and an anti-CTLA-4 antagonist.

26. The pharmaceutical composition for use of any one of claims 1 to 24, wherein the anti-PD-⅟PD-L1 antagonist comprises an antibody or antigen-binding fragment thereof that specifically binds a target protein selected from programmed death 1 (PD-1; an “anti-PD-1 antibody”) or programmed death ligand 1 (PD-L1; an “anti-PD-L1 antibody).

27. The pharmaceutical composition for use of any one of claims 1 to 26, wherein the anti-PD-⅟PD-L1 antagonist comprises an anti-PD-1 antibody.

28. The pharmaceutical composition for use of claim 26 or 27, wherein the anti-PD-1 antibody comprises nivolumab or pembrolizumab.

29. The pharmaceutical composition for use of any one of claims 1 to 26, wherein the anti-PD-⅟PD-L1 antagonist comprises an anti-PD-L1 antibody.

30. The pharmaceutical composition for use of claim 29, wherein the anti-PD-L1 antibody comprises avelumab, atezolizumab, or durvalumab.

31. The pharmaceutical composition for use of any one of claims 3 to 13 and 15 to 30, wherein the anti-CTLA-4 antagonist comprises an antibody or antigen-binding fragment thereof that specifically binds cytotoxic T-lymphocyte-associated protein 4 (CTLA-4; an “anti-CTLA-4 antibody”).

32. The pharmaceutical composition for use of claim 31, wherein the anti-CTLA-4 antibody comprises ipilimumab.

33. A method of treating a cancer in a human subject, comprising administering an anti-PD-⅟anti-PD-L1 antagonist to a subject, wherein the subject is identified as having a tumor exhibiting:
(i) an excluded CD8 localization phenotype; and
(ii) a negative PD-L1 expression status.

34. The method of claim 33, further comprising administering an anti-CTLA-4 antagonist.

35. The method of claim 33 or 34, wherein the excluded CD8 localization phenotype is measured by detecting CD8 expression in a tumor sample obtained from the subject.

36. The method of any one of claims 33 to 35, wherein the excluded CD8 localization phenotype is measured by staining the tumor sample with an antibody or an antigen-binding portion thereof that binds CD8.

37. The method of any one of claims 33 to 36, wherein the CD8 localization is measured by staining the tumor sample with an antibody or an antigen-binding portion thereof that binds CD8 followed by classification of the CD8 localization in the tumor sample;
wherein the classification is performed by a method comprising;
receiving, by at least one processor of a computing device, a plurality of histology images of tumor samples in a plurality of patients;
performing, by the at least one processor, an image analysis of the plurality of histology images to obtain a c CD8+ T-cell abundance in the tumor parenchyma and stroma in each of the plurality of histology images;
training, by the at least one processor, a machine learning algorithm using results of the image analysis and the CD8+ T-cell abundance in the tumor parenchyma and stroma;
generating, by the at least one processor, a machine learning feature space comprising a plurality of classifications based on the training; and
identifying, by the at least one processor, boundaries between the plurality of classifications in the machine learning feature space.

38. A method of identifying a human subject suitable for an anti-PD-⅟PD-L1 antagonist therapy, comprising (i) measuring an expression of PD-L1 in a tumor sample obtained from the subject, and (ii) measuring CD8 localization in the tumor sample;
wherein the CD8 localization is measured by staining the tumor sample with an antibody or an antigen-binding portion thereof that binds CD8 followed by classification of the CD8 localization in the tumor sample;
wherein the classification is performed by a method comprising:
receiving, by at least one processor of a computing device, a plurality of histology images of tumor samples in a plurality of patients;
performing, by the at least one processor, an image analysis of the plurality of histology images to obtain a CD8+ T-cell abundance in the tumor parenchyma and stroma in each of the plurality of histology images;
training, by the at least one processor, a machine learning algorithm using results of the image analysis and the CD8+ T-cell abundance in the tumor parenchyma and stroma;
generating, by the at least one processor, a machine learning feature space comprising a plurality of classifications based on the training; and
identifying, by the at least one processor, boundaries between the plurality of classifications in the machine learning feature space.

39. The method of claim 37 or 38, wherein performing the image analysis of the plurality of histology images comprises applying an artificial neural network to the plurality of histology images.

40. The method of claim 39, wherein the machine-learning algorithm comprises a random forest classifier algorithm.

41. The method any one of claims 37 to 40, wherein the CD8+ T-cell abundance comprises a graphical representation of a relationship between percentages of the stromal CD8+ T-cells and percentages of the parenchymal CD8+ T-cells with respect to the total number of T-cells present in each of the plurality of histology images.

42. The method of claim 41, further comprising: applying, by the at least one processor of the computing device, a polar coordinate transformation of the graphical representation, resulting in a polar plot; and using the polar plot to train the machine learning algorithm.

43. The method of any one of claims 37 to 42, wherein the plurality of classifications comprises inflamed, desert, excluded, or balanced.

44. The method of any one of claims 37 to 47, further comprising determining a classification for each of the plurality of histology images based on the machine learning feature space.

45. The method of claim 44, further comprising validating results from the machine learning feature space by comparing a label for each of the plurality of histology images obtained by at least one pathologist to the classification for each of the plurality of histology images.

46. The method of any one of claims 37 to 45, further comprising: receiving, by the at least one processor of the computing device, an additional histology image; performing an additional image analysis of the additional histology image and obtaining an additional CD8+ T-cell abundance in the tumor parenchyma and stroma in the additional histology image; applying the machine learning algorithm to results from the additional image analysis and the additional CD8+ T-cell abundance; and determining a classification for the additional histology image based on the machine learning feature space.

47. The method of any one of claims 38 to 47, further comprising administering the anti-PD-⅟PD-L1 antagonist to a subject identified as having an excluded CD8 localization phenotype and a PD-L1 negative tumor.

48. The method of claim 47, further comprising administering an anti-CTLA-4 antagonist.

49. The method of any one of claims 33 to 48, wherein the anti-PD-⅟PD-L1 antagonist comprises an antibody or antigen-binding fragment thereof that specifically binds a target protein selected from programmed death 1 (PD-1; an “anti-PD-1 antibody”) or programmed death ligand 1 (PD-L1; an “anti-PD-L1 antibody”).

50. The method of any one of claims 33 to 49, wherein the anti-PD-⅟PD-L1 antagonist is an anti-PD-1 antibody.

51. The method of claim 49 or 50, wherein the anti-PD-1 antibody comprises nivolumab or pembrolizumab.

52. The method of any one of claims 33 to 49, wherein the anti-PD-⅟PD-L1 antagonist comprises an anti-PD-L1 antibody.

53. The method of claim 52, wherein the anti-PD-L1 antibody comprises avelumab, atezolizumab, or durvalumab.

54. The method of any one of claims 34 to 37 and 39 to 53, wherein the anti-CTLA-4 antagonist comprises an antibody or antigen-binding fragment thereof that specifically binds cytotoxic T-lymphocyte-associated protein 4 (CTLA-4; an “anti-CTLA-4 antibody”).

55. The method of claim 54, wherein the anti-CTLA-4 antibody comprises ipilimumab.

56. The pharmaceutical composition for use of any one of claims 1 to 32, or the method of any one of claims 33 to 55, wherein the tumor is derived from a cancer selected from the group consisting of hepatocellular cancer, gastroesophageal cancer, melanoma, bladder cancer, lung cancer, kidney cancer, head and neck cancer, colon cancer, pancreatic cancer, prostate cancer, ovarian cancer, urothelial cancer, colorectal cancer, and any combination thereof.

57. The pharmaceutical composition for use of any one of claims 1 to 32 and 56, or the method of any one of claims 33 to 56, wherein the tumor is relapsed.

58. The pharmaceutical composition for use of any one of claims 1 to 32 and 56, or the method of any one of claims 33 to 56, wherein the tumor is refractory.

59. The pharmaceutical composition for use of any one of claims 1 to 32 and 56 to 58, or the method of any one of claims 33 to 58, wherein the tumor is locally advanced.

60. The pharmaceutical composition for use of any one of claims 1 to 32 and 56 to 58, or the method of any one of claims 33 to 58, wherein the tumor is metastatic.

61. The pharmaceutical composition for use of any one of claims 1 to 32 and 56 to 60, or the method of any one of claims 33 to 60, wherein the administering treats the tumor.

62. The pharmaceutical composition for use of any one of claims 1 to 32 and 56 to 61, or the method of any one of claims 33 to 61, wherein the administering reduces the size of the tumor.

63. The pharmaceutical composition or method of claim 62, wherein the size of the tumor is reduced by at least about 10%, about 20%, about 30%, about 40%, or about 50% compared to the tumor size prior to the administration.

64. The pharmaceutical composition for use of any one of claims 1 to 32 and 56 to 63, or the method of any one of claims 33 to 63, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

65. The pharmaceutical composition for use of any one of claims 1 to 32 and 56 to 64, or the method of any one of claims 33 to 64, wherein the subject exhibits stable disease after the administration.

66. The pharmaceutical composition for use of any one of claims 1 to 32 and 56 to 64, or the method of any one of claims 33 to 64, wherein the subject exhibits a partial response after the administration.

67. The pharmaceutical composition for use of any one of claims 1 to 32 and 56 to 66, or the method of any one of claims 33 to 66, wherein the subject exhibits a complete response after the administration.

68. A kit for treating a subject afflicted with a tumor, the kit comprising:
(a) an anti-PD-⅟PD-L1 antagonist; and
(b) instructions for using the anti-PD-⅟PD-L1 antagonist according to the method of any one of claims 34 to 69.

69. The kit of claim 68, wherein the anti-PD-⅟PD-L1 antagonist comprises an anti-PD-1 antibody.

70. The kit of claim 68, wherein the anti-PD-⅟PD-L1 antagonist comprises an anti-PD-L1 antibody.

71. The kit of any one of claims 68 to 70, further comprising an anti-CTLA-4 antagonist.

72. The kit of claim 71, wherein the anti-CTLA-4 agonist comprises and anti-CTLA-4 antibody.

73. The pharmaceutical composition for use of any one of claims 1 to 32 and 56 to 66, or the method of any one of claims 33 to 66, wherein the subject exhibits less severe adverse events, as compared to a subject that does not exhibit an excluded CD8 localization phenotype.

74. The pharmaceutical composition for use of any one of claims 1 to 32 and 56 to 66, or the method of any one of claims 33 to 66, wherein the subject does not exhibit an adverse event more severe than a grade 1 adverse event, more severe than a grade 2 adverse event, or more severe than a grade 3 adverse event.

75. The pharmaceutical composition for use of any one of claims 1 to 32 and 56 to 66, or the method of any one of claims 33 to 66, wherein the subject exhibits fewer adverse events of grade 3 or more severe, as compared to a subject that does not exhibit an excluded CD8 localization phenotype.
